(19) United States
(12) Patent Application Publication
(10) Pub. No.: US 2022/0084688 A1
(43) Pub. Date: Mar. 17, 2022

(54) Infectious Disease Tracking Platform

(71) Applicant: Board of Regents, The University of Texas System, Austin, TX (US)

(72) Inventors: Gulshan Sharma, League City, TX (US); Peter McCaffrey, Galveston, TX (US); Revathi Sellappan, League City, TX (US)

(21) Appl. No.: 17/474,850

(22) Filed: Sep. 14, 2021

Related U.S. Application Data
(60) Provisional application No. 63/078,678, filed on Sep. 15, 2020.

(51) Int. Cl. G16H 50/80; G16H 50/70; G16H 50/20; G16H 40/20; G16H 10/60; G16H 10/40; G06F 16/245; G06F 16/23

(52) U.S. Cl. CPC G16H 50/80; G16H 50/70; G16H 50/20; G06F 16/2379; G16H 10/60; G16H 10/40; G06F 16/245; G16H 40/20

(57) ABSTRACT

A system and method of tracking a disease includes providing an input/output interface, a memory, a staging database, a relational database and one or more processors communicably coupled to the input/output interface, the memory, the staging database and the relational database, obtaining a first data corresponding to a first time period for the disease from one or more data sources, storing the first data in the staging database, obtaining a second data corresponding to a second time period for the disease from the data sources, wherein the second time period is different than the first time period, storing the second data in the staging database, processing the first and second data stored in the staging database using a disease curated data model, storing the processed first and second data in the relational database, and tracking the disease based on a plurality of metrics using the processed first and second data.

### CROSS-REFERENCE TO RELATED APPLICATIONS

This non-provisional Patent Application claims priority to U.S. Provisional Patent Application Ser. No. 63/078,678, filed on Sep. 15, 2020, entitled “Infectious Disease Tracking Platform” the contents of which is incorporated by reference herein in its entirety.

### TECHNICAL FIELD OF THE INVENTION

The present invention relates generally to the field of information systems, more specifically, information systems relating to tracking infectious disease.

### INCORPORATION-BY-REFERENCE OF MATERIALS FILED ON COMPACT DISC

None.

### STATEMENT OF FEDERALLY FUNDED RESEARCH

None.

### BACKGROUND OF INVENTION

At present, there are not many existing, commercially available enterprise COVID dashboards. Among those few commercial approximations that do exist is Epic System's Pulse dashboard, which faces certain key limitations such as difficulty in modification, lack of control over visualization, and limitation exclusively to data within the Epic medical record.

### SUMMARY OF THE INVENTION

In one embodiment of the present invention, a system for tracking a disease includes an input/output interface, a memory, a staging database, one or more relational databases and one or more processors communicably coupled to the input/output interface, the memory, the staging database and the one or more relational databases. The memory, the staging database and/or the one or more relational databases can be local, remote or distributed. Likewise, the one or more processors can be local, remote or distributed. The staging database and/or the one or more relational databases can be linked to and access data from other data sources, which can be local, remote or distributed. The one or more processors obtain a first data corresponding to a first time period for the disease from the one or more data sources, store the first data in the staging database, obtain a second data corresponding to a second time period for the disease from the one or more data sources, wherein the second time period different than the first time period, store the second data in the staging database, process the first and second data stored in the staging database using a disease curated data model, store the processed first and second data in the relational database, and track the disease based on a plurality of metrics using the processed first and second data.

In one aspect, the one or more processors track the disease by further displaying one or more data visualizations based on one or more of the plurality of metrics or the processed first and second data. In another aspect, the one or more processors automatically send one or more alerts, guidelines, orders, or recommendations based one or more of the plurality of metrics. In another aspect, the one or more processors automatically allocate or order one or more resources based on one or more of the plurality of metrics. In another aspect, the one or more processors use one or more of the plurality of metrics to modify clinic operations, testing protocols, resource allocation or inventory management. In another aspect, the one or more processors predict one or more metrics at a future time period using the processed first and second data and the disease curated data model.

In another aspect, the one or more processors receive a query from a remote communications or processing device, update one or more of the plurality of metrics based on the query, and provide the updated metrics to the remote communications or processing device. In another aspect, the one or more processors 110 periodically update the first data or the second data, and track the disease based on the updated first data or the updated second data. In another aspect, the first data or the second data comprises new test orders, test results, admissions, locations, discharges, deaths, patient bed occupancy, ICU length of stay, or ventilator usage. In some aspects, the one or more metrics comprise patient metrics, specimen metrics, facility metrics or employee metrics. In another aspect: the patient metrics comprise positive patients, patients tested, admissions, persons under investigation, discharges, deaths, patient demographics, or patient type; the specimen metrics comprise submitters, specimens received, specimens collected, specimens resulted, specimens positive, specimens negative or test turn around time; the facility metrics comprise bed occupancy, occupancy type, length of stay, ventilator usage, discharged, death, transfer; or the employee metrics comprise employees tested, employees tested positive, employee demographics, employees currently positive, employees returned to work, employees admitted, employees tested due to screening or employees tested due to symptomatic.

In one embodiment of the present invention, a computerized method of tracking a disease includes providing an input/output interface, a memory, a staging database, a relational database and one or more processors communicably coupled to the input/output interface, the memory, the staging database and the relational database, obtaining a first data corresponding to a first time period for the disease from one or more data sources, storing the first data in the staging database, obtaining a second data corresponding to a second time period for the disease from the one or more data sources, wherein the second time period is different than the first time period, storing the second data in the staging database, processing the first and second data stored in the staging database using a disease curated data model, storing the processed first and second data in the relational database, and tracking the disease based on a plurality of metrics using the processed first and second data.

In one aspect, the first time period is hourly and the second time period is daily. In another aspect, tracking the disease further comprises displaying one or more of the plurality of metrics. In another aspect, tracking the disease further comprises displaying one or more data visualizations based on one or more of the plurality of metrics or the processed first and second data. In another aspect, the method further comprises automatically sending one or more alerts, guidelines, orders, or recommendations based one or more of the plurality of metrics. In another aspect, the method further comprises automatically allocating or ordering one or more resources based on one or more of the plurality of metrics. In another aspect, the method further comprises using one or more of the plurality of metrics to modify clinic operations, testing protocols, resource allocation or inventory management. In another aspect, the method further comprises predicting one or more metrics at a future time period using the processed first and second data and the disease curated data model. In another aspect, the method further comprises receiving a query from a remote communications or processing device, updating one or more of the plurality of metrics based on the query, and providing the updated metrics to the remote communications or processing device. In another aspect, the method further comprises periodically updating the first data or the second data, and tracking the disease based on the updated first data or the updated second data. In another aspect, the first data or the second data comprises new test orders, test results, admissions, locations, discharges, deaths, patient bed occupancy, ICU length of stay, or ventilator usage. In another aspect, the one or more metrics comprise patient metrics, specimen metrics, facility metrics or employee metrics. In another aspect: the patient metrics comprise positive patients, patients tested, admissions, persons under investigation, discharges, deaths, patient demographics, or patient type; the specimen metrics comprise submitters, specimens received, specimens collected, specimens resulted, specimens positive, specimens negative or test turn around time; the facility metrics comprise bed occupancy, occupancy type, length of stay, ventilator usage, discharged, death, transfer; or the employee metrics comprise employees tested, employees tested positive, employee demographics, employees currently positive, employees returned to work, employees admitted, employees tested due to screening or employees tested due to symptomatic.

Various embodiments of the present invention is described in detail below with reference to the accompanying drawings.

### BRIEF DESCRIPTION OF THE DRAWINGS

The above and further advantages of the invention may be better understood by referring to the following descriptions in conjunction with the accompanying drawings, in which:

FIG. 1 is a block diagram of a system according to an embodiment of the current invention;

FIG. 2 is a flow chart of a method in according to an embodiment of the current invention;

FIG. 3 is an illustrative schematic of data flow within an infectious disease tracking platform according to an embodiment of the current invention;

FIG. 4 is an illustrative schematic of data flow within an infectious disease tracking platform according to an embodiment of the current invention;

FIG. 5 is a dashboard page of Assessment of Laboratory Testing Bandwidth and Modification of Specimen Pickup Times and Couriers according to an embodiment of the current invention;

FIG. 6 is dashboard page of Identification of Inpatient Testing Backlog and Prioritization of Patients Total Testing, and Adequacy of Public Health Monitoring according to an embodiment of the current invention;

FIG. 7 is a dashboard page of Mechanical Ventilator Capacity Planning according to an embodiment of the current invention;

FIG. 8 is a dashboard page of Employee Transmission and Testing Protocols according to an embodiment of the current invention;

FIG. 9 is a dashboard page of Hospital Admission and Patient Follow Up according to an embodiment of the current invention;

FIG. 10 is a dashboard page of Monitoring and Guidance for Outpatient Testing Protocols according to an embodiment of the current invention;

FIGS. 11-50 depict various non-limiting examples of dashboards, charts, graphs and other data visualization according to embodiments of the current invention;

FIG. 51 lists various non-limiting metric definitions according to embodiments of the current invention; and

FIGS. 52A-52B list various non-limiting metrics, sources and modules according to embodiments of the current invention.

### DETAILED DESCRIPTION OF THE INVENTION

The current invention now will be described more fully hereinafter with reference to the accompanying drawings, which illustrate embodiments of the invention. This invention may, however, be embodied in many different forms and should not be construed as limited to the illustrated embodiments set forth herein. The examples relate to COVID-19, but embodiments of the present invention can be used for other disease. Moreover, the present invention is not limited to use with diseases or in a healthcare environment. For example, embodiments of the present invention can be used to track the spread or containment of a phenomenon that has detectable metrics. The embodiments described herein are provided so that this disclosure will be thorough and complete, and will fully convey the scope of the invention to those skilled in the art.

Various embodiments of the present invention are beneficial to hospitals, health departments and cities. All healthcare institutions are impacted in some way by COVID-19 typically in the form of decreased bed occupancy and revenue. Thus, there is an impetus to re-open as quickly as possible while still safeguarding patients. This cannot be done without close monitoring of the presence and growth of infection among tested patients. Thus, it is imperative that organizations can define and consistently track metrics of disease burden and bed utilization. Moreover, COVID-19 is but one of many infectious diseases that impact hospital operations. In each such case, monitoring is necessary for effective management. Like hospitals, health departments and cities have been significantly disrupted by the spread of COVID-19 with an even stronger pressure placed upon them to re-open businesses and public areas. Likewise, this cannot be accomplished without close disease tracking which likely involves collaboration with hospitals. In the case of each of these groups, constructing a monitoring system for an infectious disease can be a resource-intensive task that runs the risk of ultimately producing low-value or non-standard metrics and visualizations. The disease tracking platform described herein offers a standard solution that can be integrated with an institution's existing IT systems.

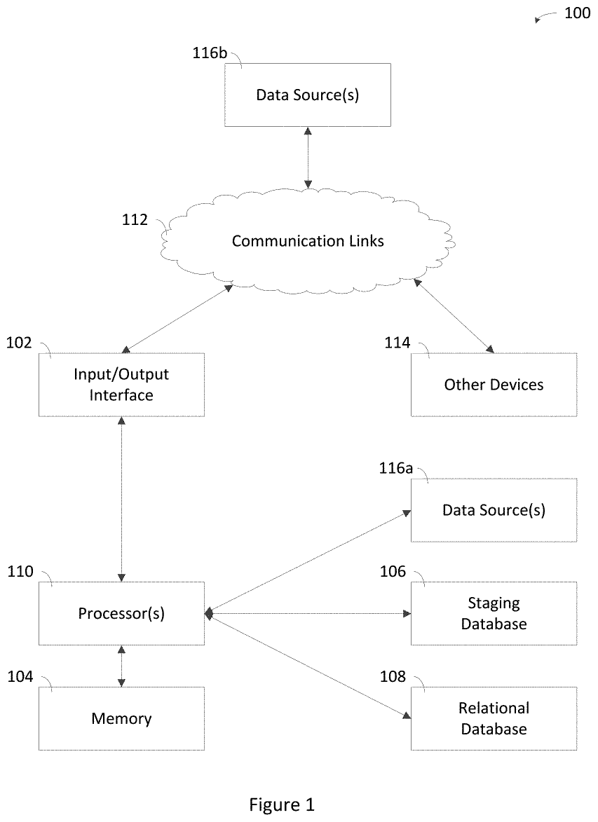

FIG. 1 is a block diagram of a system 100 for tracking a disease according to an embodiment of the current invention. The system 100 includes an input/output interface 102, a memory 104, a staging database 106, one or more relational databases 108 and one or more processors 110 communicably coupled to the input/output interface 102, the memory 104, the staging database 106 and the one or more relational databases 108. The memory 104, the staging database 106 and/or the one or more relational databases 108 can be local, remote or distributed. Likewise, the one or more processors 110 can be local, remote or distributed. The the staging database 106 and/or the one or more relational databases 108 can be linked to and access data from other data sources, which can be local, remote or distributed. The input/output interface 102 can be any mechanism for facilitating the input and/or output of information (e.g., web-based interface, touchscreen, keyboard, mouse, display, printer, etc.) Moreover, the input/output interface 102 can be a remote device communicably coupled to the one or more processors 110 via one or more communication links 112 (e.g., network(s), cable(s), wireless, satellite, etc.). The one or more communication links 112 can communicably couple the system 100 to other devices 114 (e.g., databases, remote devices, hospitals, doctors, researchers, patients, etc.). The system 100 also includes access to one or more data sources 116, which can be local data source(s) 116a and/or remote data sources 116b. The system 100 can be implemented with various devices, such as, server computers, workstation computers, laptop computers, mobile communications devices, personal data assistants, scanning devices or any other devices capable of performing the functions described herein. Note also that the system 100 may include other components not specifically described herein.

The one or more processors 110 obtain a first data corresponding to a first time period for the disease from the one or more data sources 116, store the first data in the staging database 106, obtain a second data corresponding to a second time period for the disease from the one or more data sources 116, wherein the second time period different than the first time period, store the second data in the staging database 106, process the first and second data stored in the staging database 106 using a disease curated data model, store the processed first and second data in the relational database 108, and track the disease based on a plurality of metrics using the processed first and second data.

In one aspect, the first time period is hourly and the second time period is daily. In another aspect, the one or more processors track the disease by further displaying one or more of the plurality of metrics. In another aspect, the one or more processors 110 track the disease by further displaying one or more data visualizations based on one or more of the plurality of metrics or the processed first and second data. In another aspect, the one or more processors 110 automatically send one or more alerts, guidelines, orders, or recommendations based one or more of the plurality of metrics. In another aspect, the one or more processors 110 automatically allocate or order one or more resources based on one or more of the plurality of metrics. In another aspect, the one or more processors 110 use one or more of the plurality of metrics to modify clinic operations, testing protocols, resource allocation or inventory management. In another aspect, the one or more processors 110 predict one or more metrics at a future time period using the processed first and second data and the disease curated data model.

In another aspect, the one or more processors 110 receive a query from a remote communications or processing device, update one or more of the plurality of metrics based on the query, and provide the updated metrics to the remote communications or processing device. In another aspect, the one or more processors 110 periodically update the first data or the second data, and track the disease based on the updated first data or the updated second data. In another aspect, the first data or the second data comprises new test orders, test results, admissions, locations, discharges, deaths, patient bed occupancy, ICU length of stay, or ventilator usage. In another aspect, the one or more metrics comprise patient metrics, specimen metrics, facility metrics or employee metrics. In another aspect: the patient metrics comprise positive patients, patients tested, admissions, persons under investigation, discharges, deaths, patient demographics, or patient type; the specimen metrics comprise submitters, specimens received, specimens collected, specimens resulted, specimens positive, specimens negative or test turn around time; the facility metrics comprise bed occupancy, occupancy type, length of stay, ventilator usage, discharged, death, transfer; or the employee metrics comprise employees tested, employees tested positive, employee demographics, employees currently positive, employees returned to work, employees admitted, employees tested due to screening or employees tested due to symptomatic.

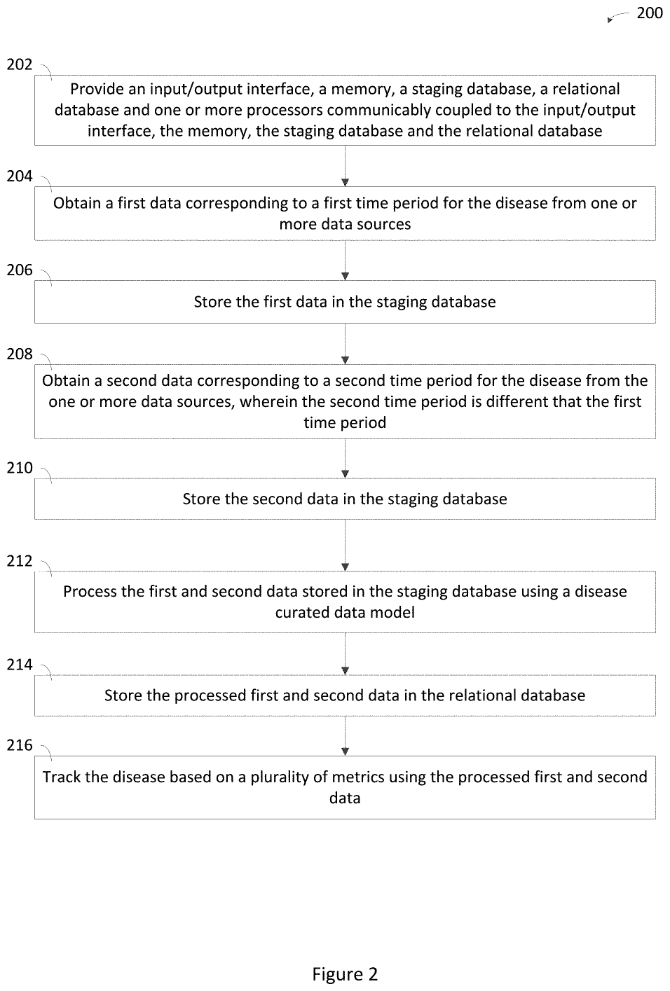

FIG. 2 is a flow chart 200 of a computerized method of tracking a disease. An input/output interface, a memory, a staging database, a relational database and one or more processors communicably coupled to the input/output interface, the memory, the staging database and the relational database are provided in block 202. A first data corresponding to a first time period for the disease is obtained from one or more data sources in block 204. The first data is stored in the staging database in block 206. A second data corresponding to a second time period for the disease is obtained from the one or more data sources in block 208, wherein the second time period is different than the first time period. The second data is stored in the staging database in block 210. The first and second data stored in the staging database are processed using a disease curated data model in block 212. The processed first and second data are stored in the relational database in block 214. The disease is tracked based on a plurality of metrics using the processed first and second data in block 216.

In one aspect, the first time period is hourly and the second time period is daily. In another aspect, tracking the disease further comprises displaying one or more of the plurality of metrics. In another aspect, tracking the disease further comprises displaying one or more data visualizations based on one or more of the plurality of metrics or the processed first and second data. In another aspect, the method further comprises automatically sending one or more alerts, guidelines, orders, or recommendations based one or more of the plurality of metrics. In another aspect, the method further comprises automatically allocating or ordering one or more resources based on one or more of the plurality of metrics. In another aspect, the method further comprises using one or more of the plurality of metrics to modify clinic operations, testing protocols, resource allocation or inventory management. In another aspect, the method further comprises predicting one or more metrics at a future time period using the processed first and second data and the disease curated data model. In another aspect, the method further comprises receiving a query from a remote communications or processing device, updating one or more of the plurality of metrics based on the query, and providing the updated metrics to the remote communications or processing device. In another aspect, the method further comprises periodically updating the first data or the second data, and tracking the disease based on the updated first data or the updated second data. In another aspect, the first data or the second data comprises new test orders, test results, admissions, locations, discharges, deaths, patient bed occupancy, ICU length of stay, or ventilator usage. In another aspect, the one or more metrics comprise patient metrics, specimen metrics, facility metrics or employee metrics. In another aspect: the patient metrics comprise positive patients, patients tested, admissions, persons under investigation, discharges, deaths, patient demographics, or patient type; the specimen metrics comprise submitters, specimens received, specimens collected, specimens resulted, specimens positive, specimens negative or test turn around time; the facility metrics comprise bed occupancy, occupancy type, length of stay, ventilator usage, discharged, death, transfer; or the employee metrics comprise employees tested, employees tested positive, employee demographics, employees currently positive, employees returned to work, employees admitted, employees tested due to screening or employees tested due to symptomatic.

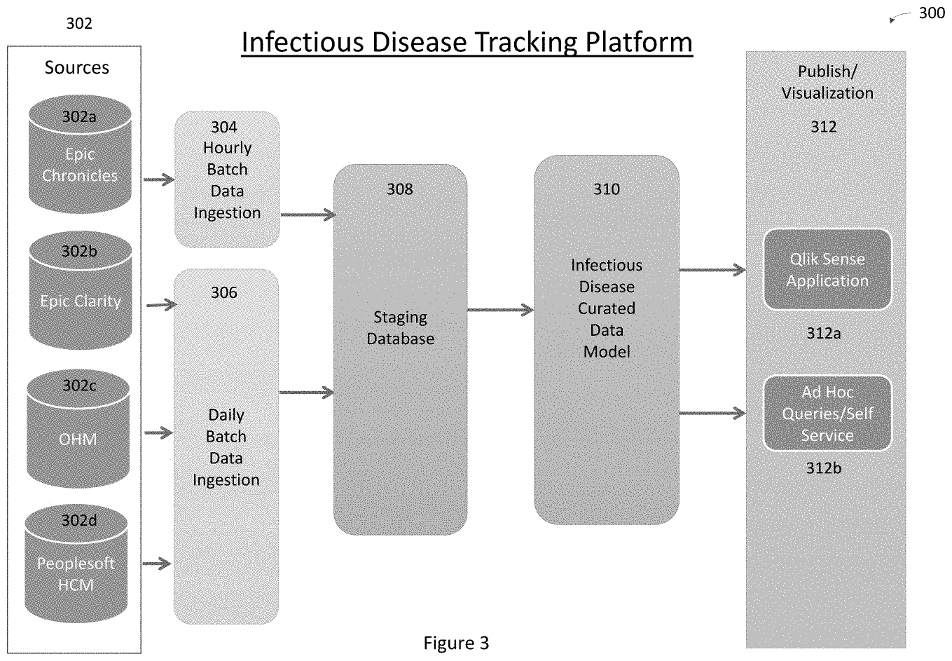

In one illustrative embodiment of the present invention shown in FIG. 3, the Tracking Platform 300 includes various data sources 302, such as Epic Chronicles 302a, Epic Clarity 302b, OHM 302c and Peoplesoft HCM 302d. Other data sources can be used. Hourly batch data 304 and daily batch data 306 are ingested from the multiple data sources 302, with the primary source being the Epic Medical Record System, and stored in a staging database 308. A curated infectious disease data model 310 (e.g., COVID or any other infectious disease) is used to organize the ingested data elements, standardize how they relate to one another and apply real world logic to support reporting. The data is then feed into robust end user applications 312, such as Qlik Sense 312a that presents specialized metrics and visualizations for monitoring COVID-19 and managing related hospital operations, and ad hoc queries/self service 312b. Importantly, this application receives data from Epic hourly to reflect new test orders, test results, admissions/locations, discharges, deaths, patient bed occupancy, ICU length of stay and ventilator usage among other items. This application serves metrics that have been formulated and implemented by UTMB for the purposes of monitoring the COVID-19 pandemic and each of which bears relevance to disease surveillance and management. The application also includes a collection of metrics definitions in both technical and plain text.

This application was built to provide visibility to the Healthsystem during the management of COVID-19. The novelty and rapidity of this disease process have required hospitals to establish monitoring systems very quickly and to use those systems to define best practice where historical guidelines are not present. Thus, this application serves as a central source of truth and guidance for Healthsystem leadership as they must make decisions about clinic operations, testing protocols, ventilator acquisition, and many other issues urgently and continuously. Lastly, as COVID-19 is an evolving situation, the existence of the database that supports this system's Qlik dashboards allows for ad-hoc querying and interrogation with real time COVID testing data. Moreover, this application implements a collection of salient metrics using extensible tools such as SQL Server and Qlik Sense, allowing for it to adapt and grow within healthcare organizations.

Finally, as UTMB has performed almost 70,000 COVID tests at the time of this writing, this dashboard system and its metrics are informed by a larger volume of COVID tests than most institutions, giving UTMB the ability to provide baseline metrics and predictions that other institutions would not be able to power.

Notably, as this system establishes a direct connection to Epic across various modules including laboratory, admissions and bed occupancy, demographics, and other information, this system is generalizable beyond COVID. An example of this is the system's specimens/test results monitoring page, which describes core operations of the clinical laboratory including specimens received, specimens resulted, pending lists, and instrument performance. These are utterly generalizable to any combination or collection of tests performed.

In summary, this disclosure describes a technical system for collecting and presenting a broad collection of key metrics describing an infectious disease process and allowing for its monitoring and management. This system has been purposed to meet the needs of COVID-19 but can be readily reconfigured for any infectious disease.

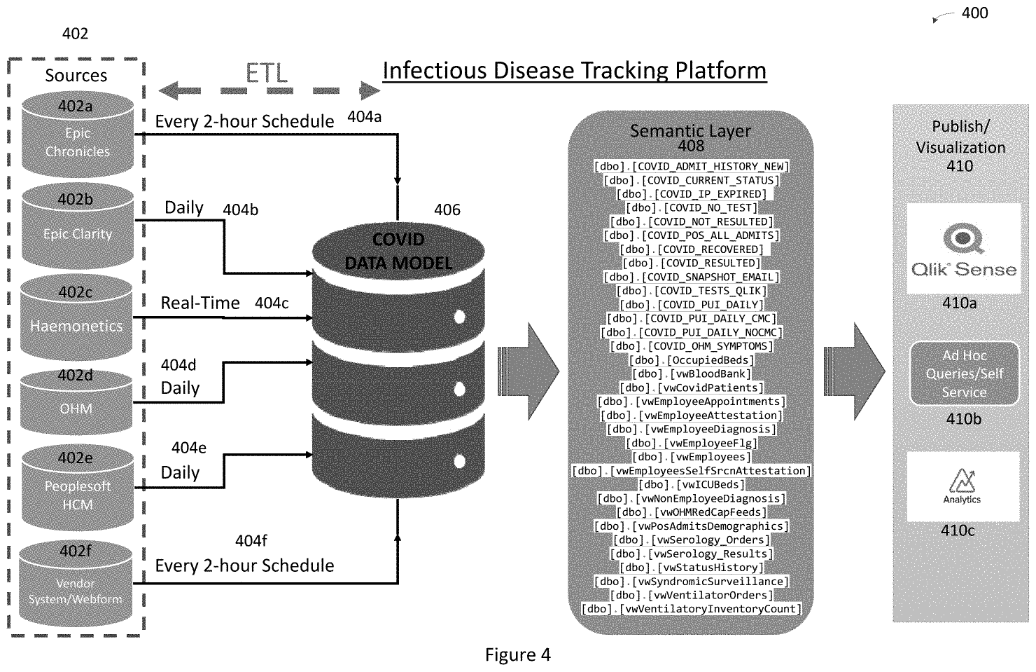

In another illustrative embodiment of the present invention shown in FIG. 4, the Tracking Platform 400 includes various data sources 402, such as Epic Chronicles 402a, Epic Clarity 402b, Haemonetics 402c, OHM 402d, Peoplesoft HCM 402d and Vendor System/Webform 402f. Other data sources can be used. Data 404a is ingested from Epic Chronicles 402a on a two hour schedule. Data 404b is ingested from Epic Clarity 402b on a daily basis. Data 404c is ingested from Haemonetics 402c in real-time. Data 404d is ingested from OHM 402d on a daily basis. Data 404e is ingested from Peoplesoft HCM 402e on a daily basis. Data 404f is ingested from Vendor System/Webform 402f on a two hour schedule. A COVID data model 406 is used to organize the ingested data elements, standardize how they relate to one another and apply real world logic to support reporting. The data is then feed into robust end user applications 410 using semantic layer 408. The publish/visualization applications 410 may include Qlik Sense 410a, ad hoc queries/self service 410b and analytics 410c. Other applications can be used.

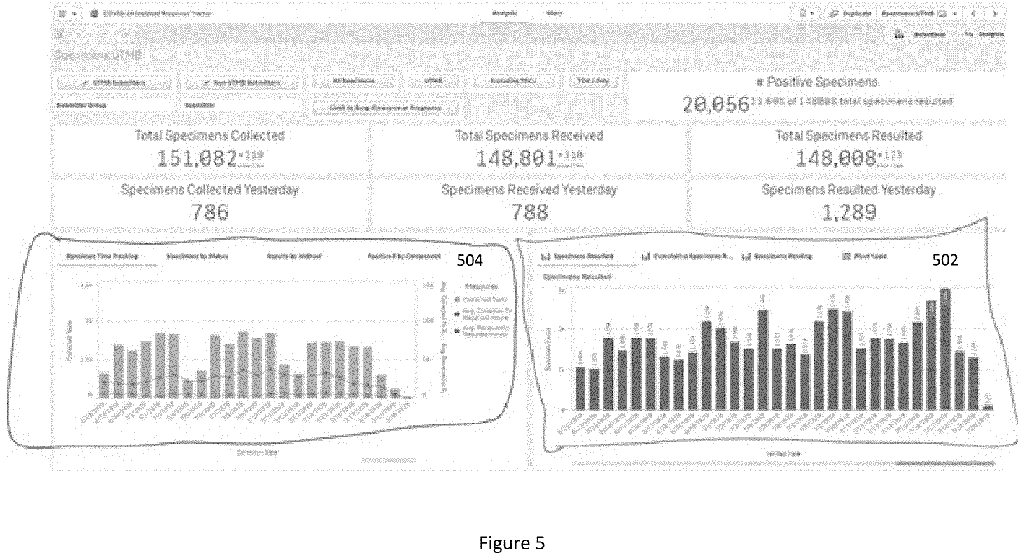

Non-limiting examples of various dashboard use cases of the current invention will now be described in reference to FIGS. 5-10.

FIG. 5 illustrates an Assessment of Laboratory Testing Bandwidth. The Specimens tab of the COVID Incident Response Dashboard includes (among a tally of tests performed) a chart depicting the size of the “pending list” each day. This pending list represents the size of un-resulted specimens left over at the end of each day (chart 502). This graph has been used to determine whether the laboratory is capable of meeting testing demand. An upward trend in this chart indicates that the lab is falling farther behind each day and is in need of more testing instruments or staff while a steady state indicates that the laboratory is meeting its current demand. This page also displays the number of positive specimens, total specimens collected, total specimens received, total specimens resulted, specimens collected yesterday, specimens received yesterday, and specimens resulted yesterday.

FIG. 5 also illustrates a Modification of Specimen Pickup Times and Couriers. In a similar way as the above example, there is a chart depicting the average elapsed time between specimen collection and arrival in the laboratory for each day (chart 504). This chart has been used to identify backlogs in specimen shipment, to motivate modification of specimen pickup times from clinic sites, and to monitor and ensure that such modifications effectively improve the collection-receiving time gap.

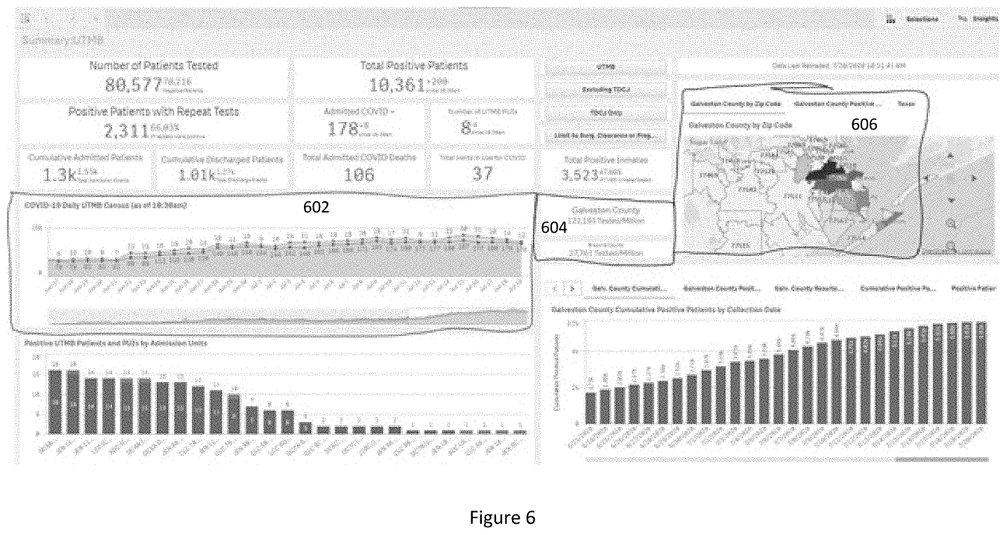

FIG. 6 illustrates an Identification of Inpatient Testing Backlog and Prioritization of Patients dashboard page. The first page of the app (the Summary page) depicts many useful metrics. One key to management of this and any other infectious disease is a daily chart showing both the number of COVID-positive patients currently admitted as well as the number of admitted patients for whom someone has ordered testing for COVID but the result has not yet been determined. This latter group are referred to as “Persons Under Investigation” (PUI) and effective triaging of COVID testing requires that inpatients be tested quickly so that they ran be ruled in or ruled out and located to the appropriate unit such that they do not risk infection of other patients. This chart has been used (chart 602) to determine whether inpatient specimens are being appropriately processed. The aim is to keep the number of active PUIs at any one time (denoted in red) as low and steady as possible. A climb in this number indicates that we are not appropriately triaging these specimens. In addition, this page displays the number of patients tested, total positive patients, positive patients with repeat tests, admitted COVID+, number of UTMB PUIs, cumulative admitted patients, cumulative discharged patients, total admitted COVID deaths, total ventilators in use for COVID, and total positive inmates.

FIG. 6 also illustrates a Total Testing and Adequacy of Public Health Monitoring. By integrating test data with patient data including demographics such as county of residence, it is possible to calculate prevalence of positive cases by County and Zip Code. Moreover, it is also possible to calculate the number of patients tested per million population across Counties (displayed numerically 604 and geographically 606). This is a core guiding metric that speaks to the adequacy of the testing efforts and informs the legitimacy of the test data as a proxy for population prevalence. This information has been used both to establish as well to monitor achievement of target testing goals. This page also displays graphs for positive UTMB patients and PUIs by admission units in the lower left, and Galveston county positive UTMB patient % by collected data.

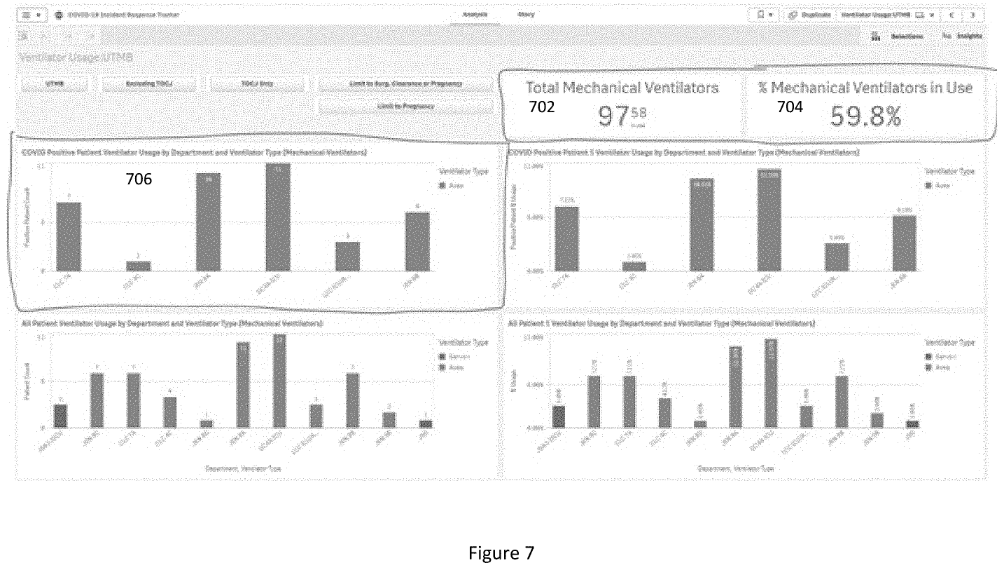

FIG. 7 illustrates a Mechanical Ventilator Capacity Planning dashboard page dedicated to Ventilator Usage that shows which and how many ventilators are in use across the hospital system 702. This also shows to what portion of the total ventilator capacity is currently in use for COVID patients 704. This page has been used to determine whether the entity currently possess enough mechanical ventilators to serve the patients. It has also been used to know which units are the most common recipients of ventilators (chart 706). This is something that helps to keep certain ventilators within COVID units and help prevent transmission within the hospital. The creation of this report requires aggregation of patient testing data with ventilator flowsheets along with the creation and dissemination of a standard protocol for documenting ventilator use in flowsheets. This page also displays graphs of all patient ventilator usage by department and ventilator type in the lower left, COVID positive patient % ventilator usage by department and ventilator type in the middle right, and all patient % ventilator usage by department and ventilator type in the lower right.

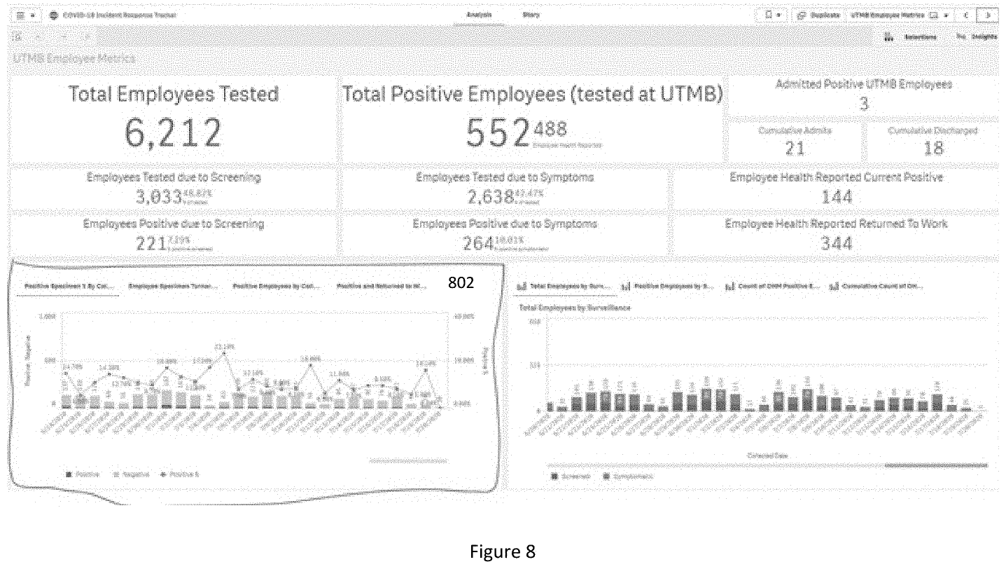

FIG. 8 illustrates an Employee Transmission and Testing Protocols dashboard page dedicated to Employee testing. This page includes many useful things among which is a chart depicting the percent positivity per day from among our tested employees (chart bounded 802). This has been very useful to monitor transmission and spill over between the COVID population and employees, determining whether there is a spike in positivity among the clinical workforce. Moreover, this page pulls in data from Employee Health clinics through a customized ingestion process and shows whether enough patients are getting tested at these sites. This is both a status monitor and a guide for how many test sites are operated, the expected testing throughput for employees, and both when and whether there is concern enough about employee transmission to modify openings or staff schedules. This page also displays the total number employees tested, total positive employees, admitted positive UTMB employees, cumulative admits, cumulative discharged, employees tested due to screening, employees positive due to screening, employees tested due to symptoms, employees positive due to symptoms, employee health reported current positive, employee health reported returned to work, and a graph of total employees by surveillance.

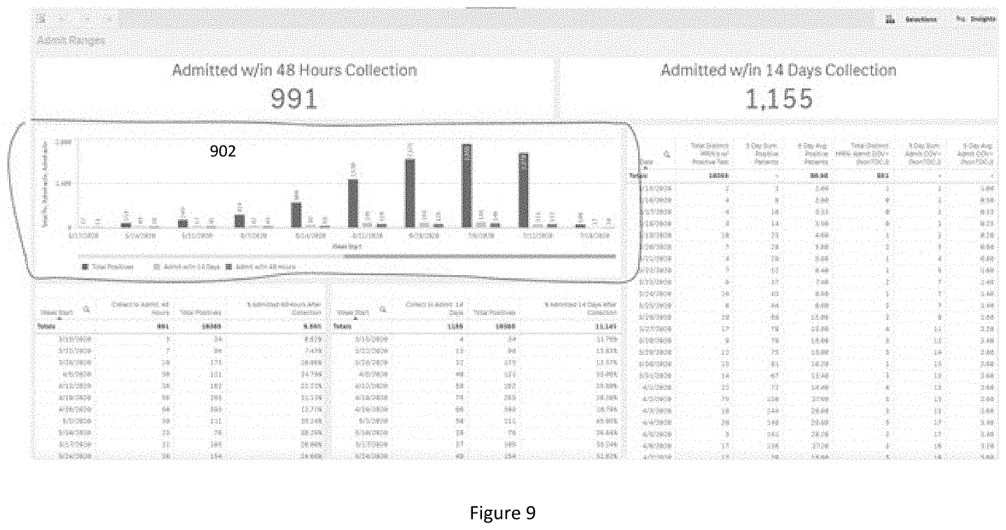

FIG. 9 illustrates a Hospital Admission and Patient Follow Up dashboard page dedicated to admission metrics. More specifically, this page tracks the number of admissions with 2 or 14 days of testing positive (chart 902). This is used to determine—for the population—what the average time is between outpatient presentation and an illness significant enough to warrant hospitalization. This is used for a variety of things such as predicting a surge of admissions but this is also used to determine how outpatient testing sites are being used and whether they are primarily seeing patients with a mild or an advanced illness. This page also displays admitted within 48 hours collection, admitted within 14 days collection, and data details.

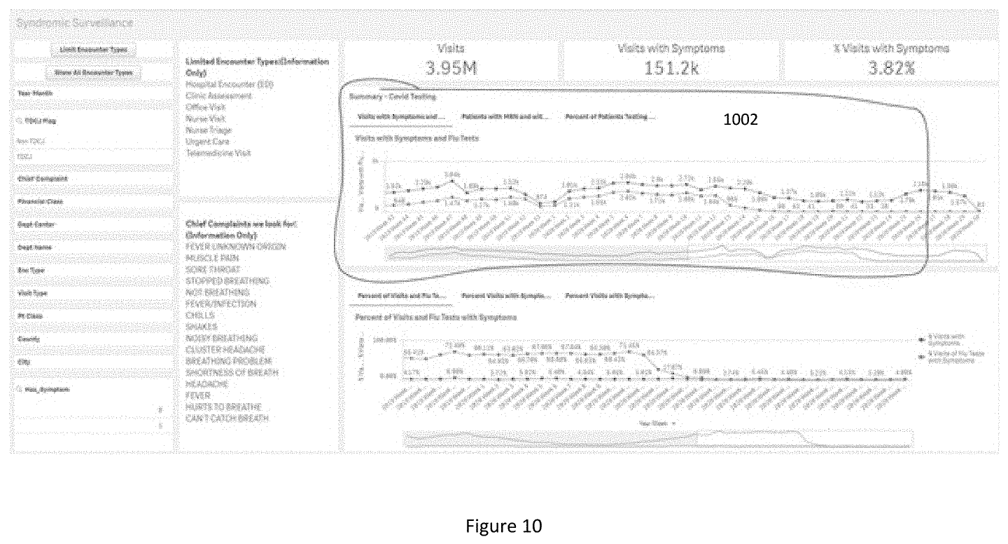

FIG. 10 illustrates a Monitoring and Guidance for Outpatient Testing Protocols dashboard page, which is “Syndromic Surveillance”. This means whether and to what extent patients who are tested have symptoms that would suggest COVID-19. There is both the ability to filter by diagnostic group or encounter class as well as a chart displaying the trend in symptomatic vs. asymptomatic visits over time (chart 1002). This is especially useful in determining site and population-specific testing protocols and whether additional questions should be added as part of a routine COVID screening appointment. The logic behind this involved custom aggregation of diagnosis codes from Epic as well as testing and order information. This page also displays a graph of percent visits and flu tests with symptoms.

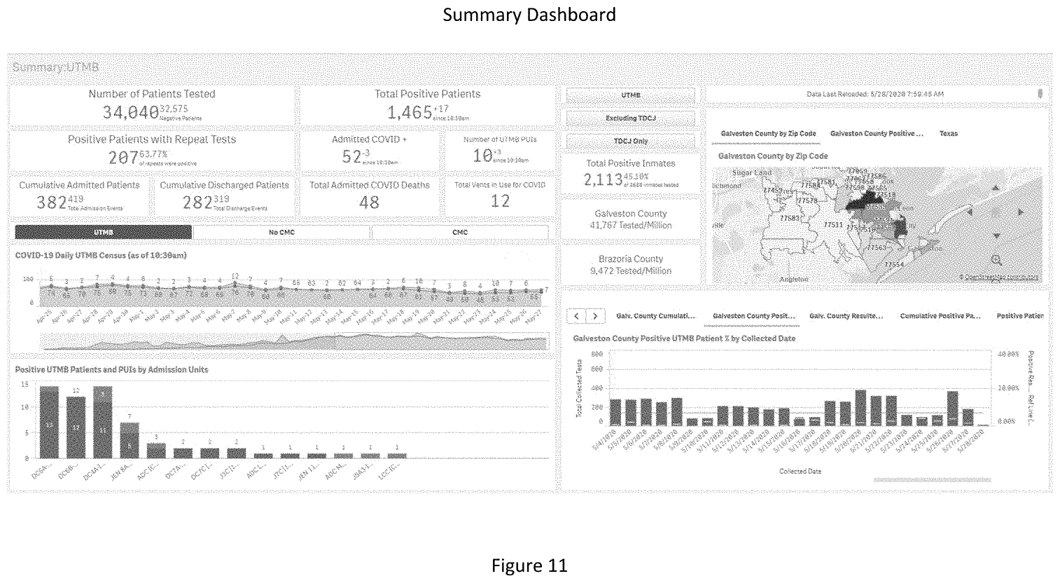

Additional non-limiting examples of various dashboards, charts, graphs and other data visualization of the current invention are shown in FIGS. 11-50. Other data visualization and combinations thereof can be used.

FIG. 11—Summary Dashboard, which is also described above in reference to FIG. 6.

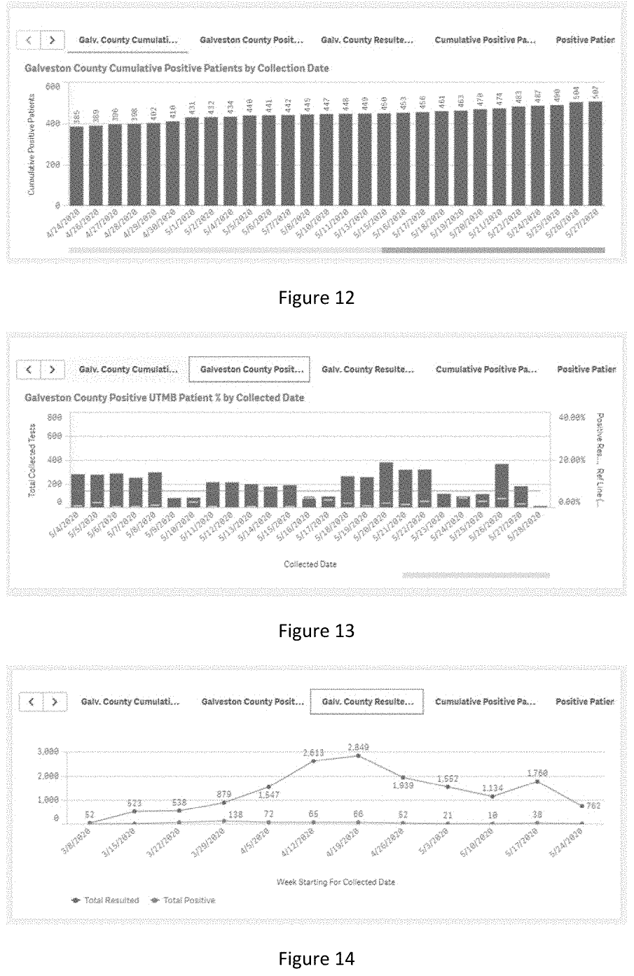

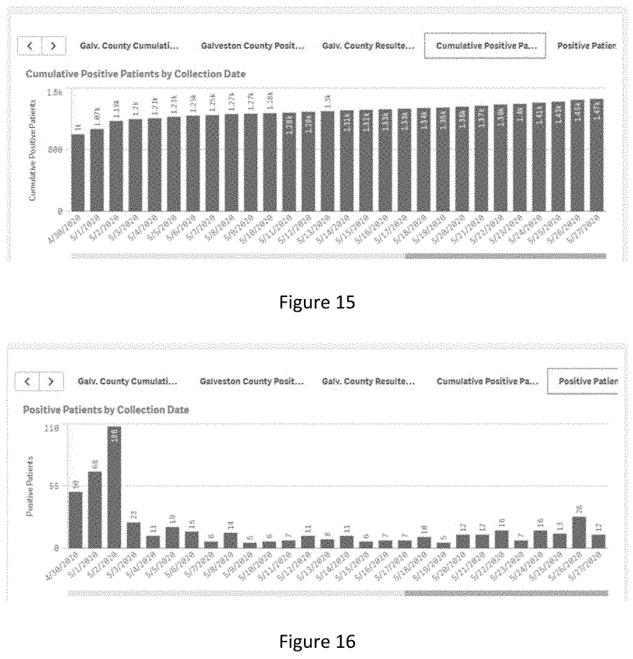

FIGS. 12-16—Summary Charts: Galveston county cumulative positive patients by collection data (FIG. 12), Galveston county positive UTMB patient % by collected data (FIG. 13); Galveston county resulted and positive (FIG. 14), cumulative positive patients by collection date (FIG. 15), and positive patients by collection date (FIG. 16).

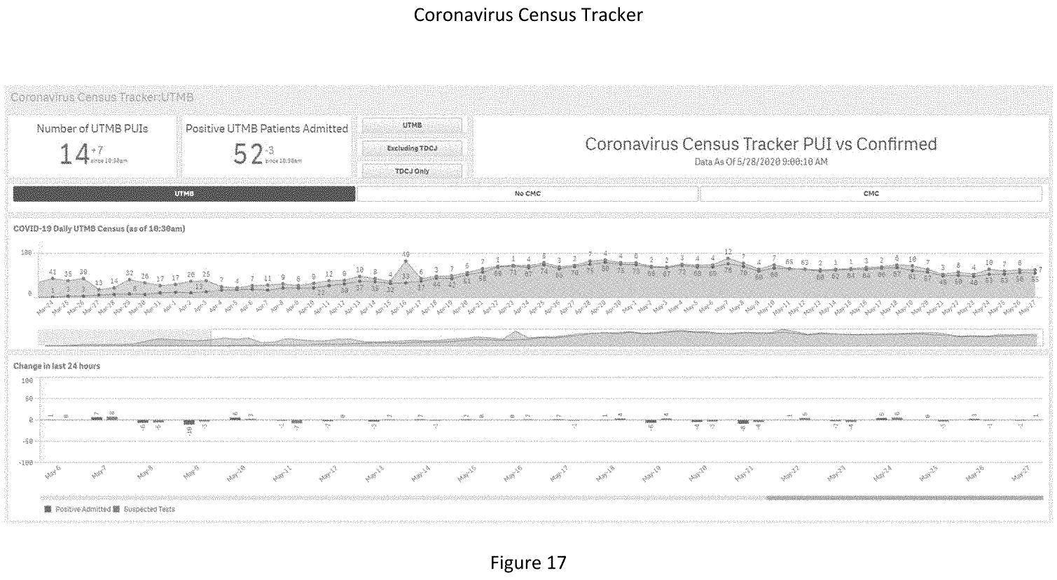

FIG. 17—Coronavirus Census Tracker Dashboard, which displays number of UTMB PUIs, positive UTMB patients admitted, and graphs of COVID-19 daily UTMB census and change in last 24 hours.

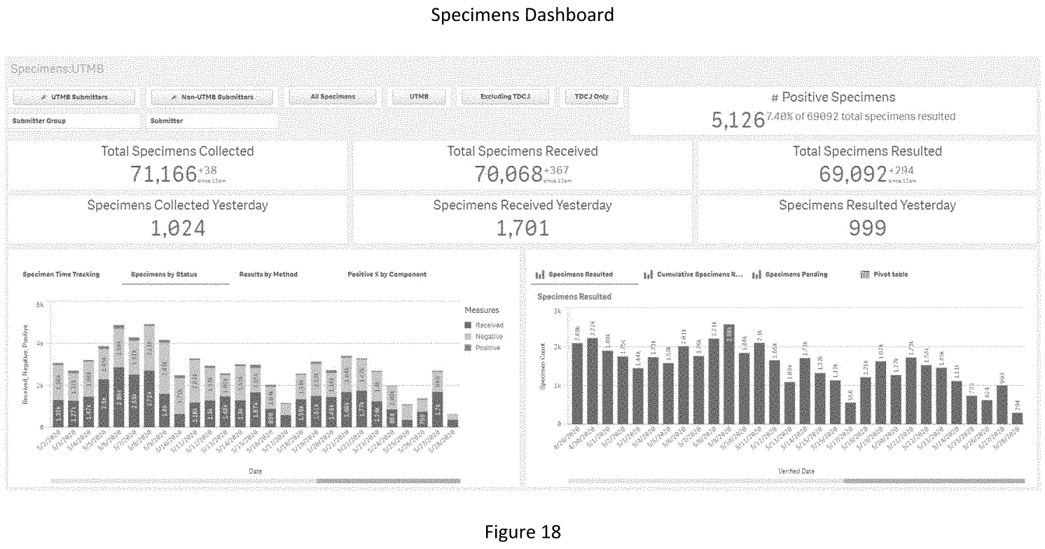

FIG. 18—Specimens Dashboard, which is also described above in reference to FIG. 5.

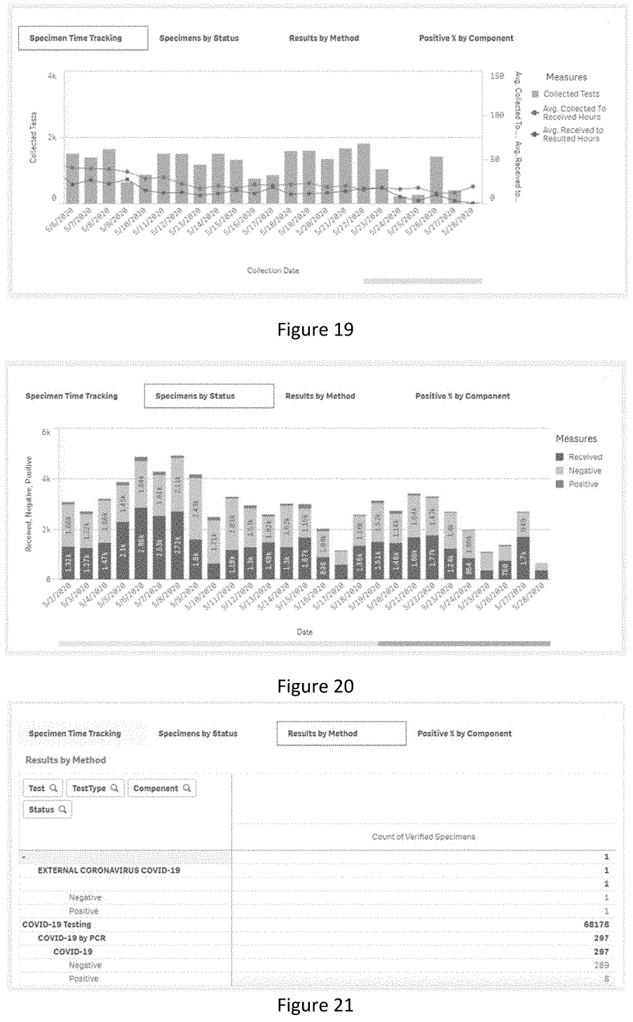

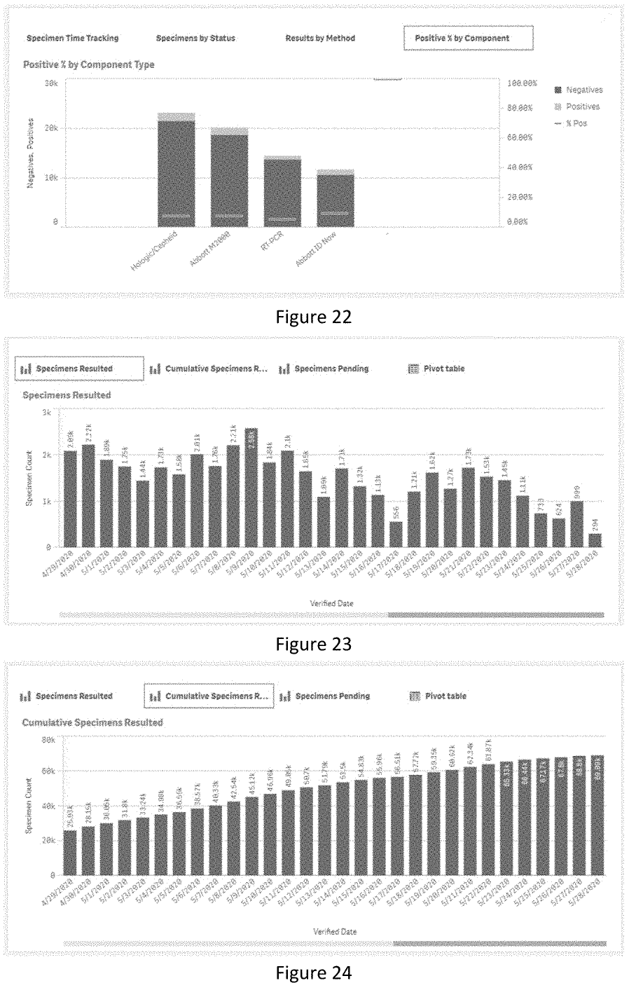

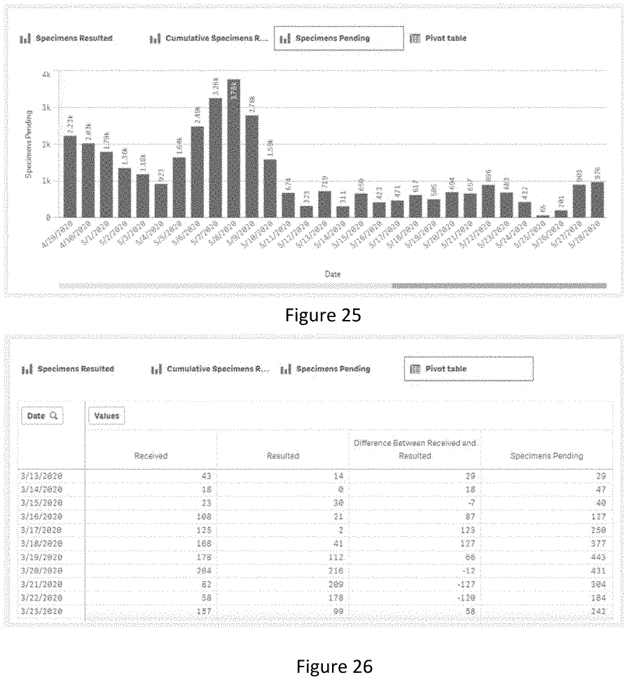

FIGS. 19-26—Specimen Charts: specimen time tracking (FIG. 19), specimens by status (FIG. 20), results by method (FIG. 21), positive % by component (FIG. 22), specimens resulted (FIG. 23), cumulative specimens resulted (FIG. 24), specimens pending (FIG. 25), and pivot table (FIG. 26).

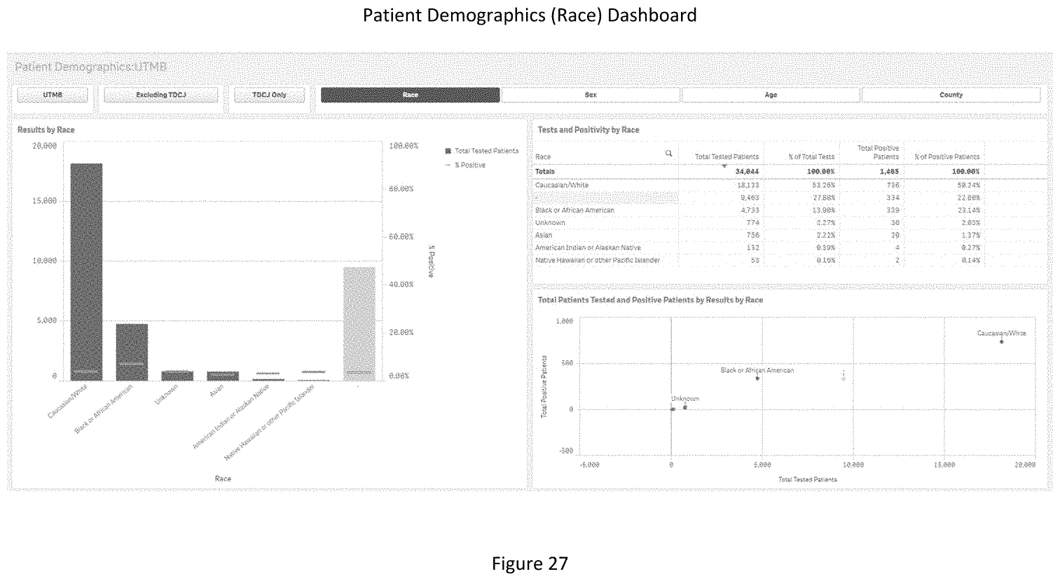

FIG. 27—Patient Demographics (Race) Dashboard, which displays a graph of results by race, tests and positivity by race data, and total patients tested and positive patients by results by race.

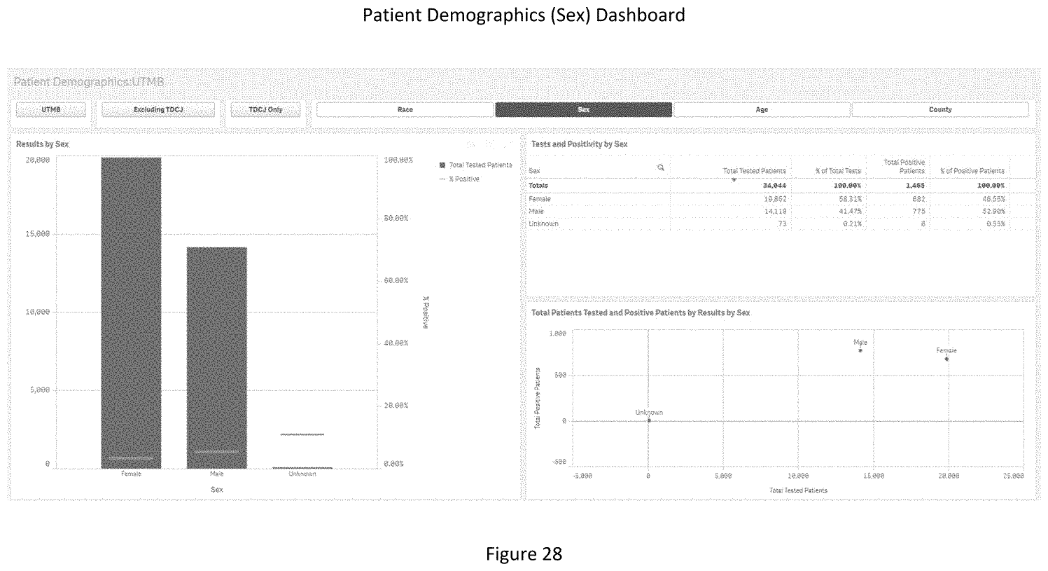

FIG. 28—Patient Demographics (Sex) Dashboard, which displays a graph of results by sex, tests and positivity by race sex, and total patients tested and positive patients by results by sex.

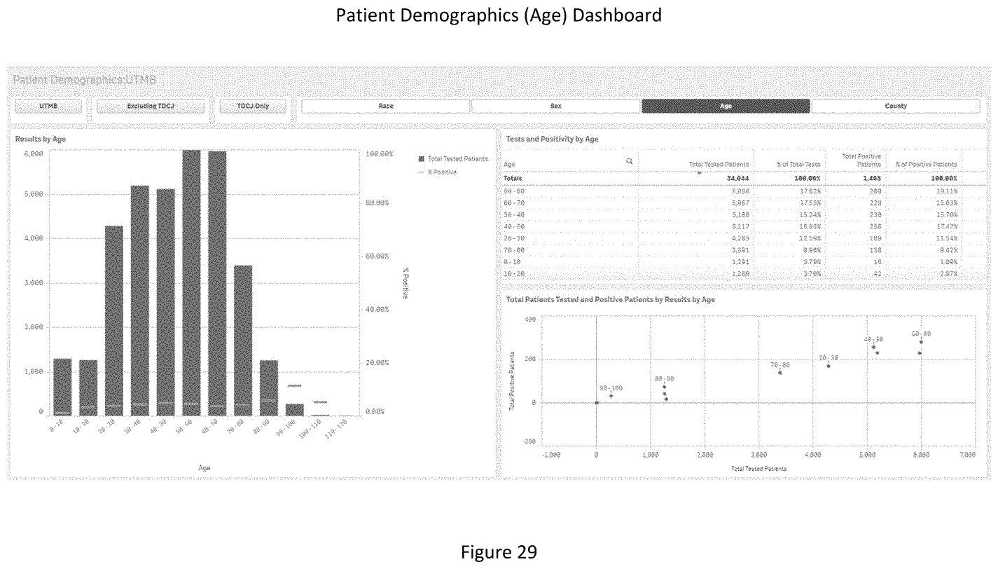

FIG. 29—Patient Demographics (Age) Dashboard, which displays a graph of results by age, tests and positivity by age data, and total patients tested and positive patients by results by age.

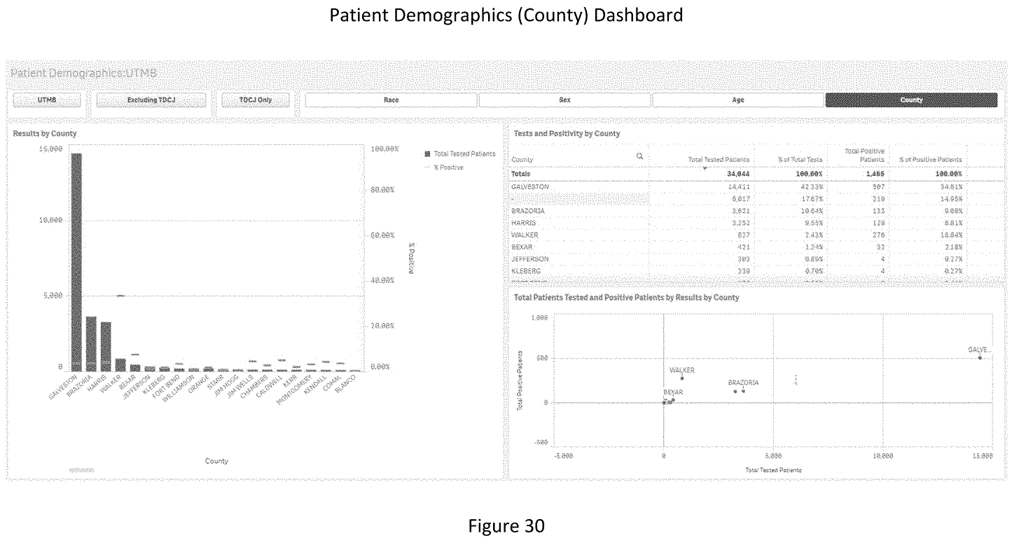

FIG. 30—Patient Demographics (County) Dashboard, which displays a graph of results by county, tests and positivity by race county, and total patients tested and positive patients by results by county.

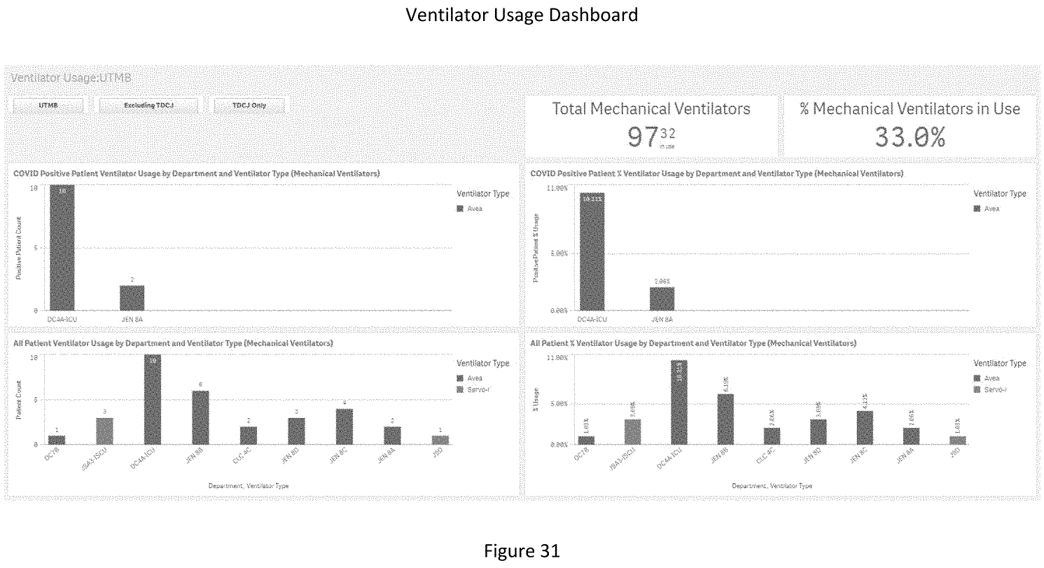

FIG. 31—Ventilator Usage Dashboard, which displays total mechanical ventilators, % mechanical ventilators in use, and graphs for COVID positive patient ventilator usage by department and ventilator type (upper left), all patient ventilator usage by department and ventilator type (lower left), COVID positive patient % ventilator usage by department and ventilator type (middle right), and all patient % ventilator usage by department and ventilator type (lower right).

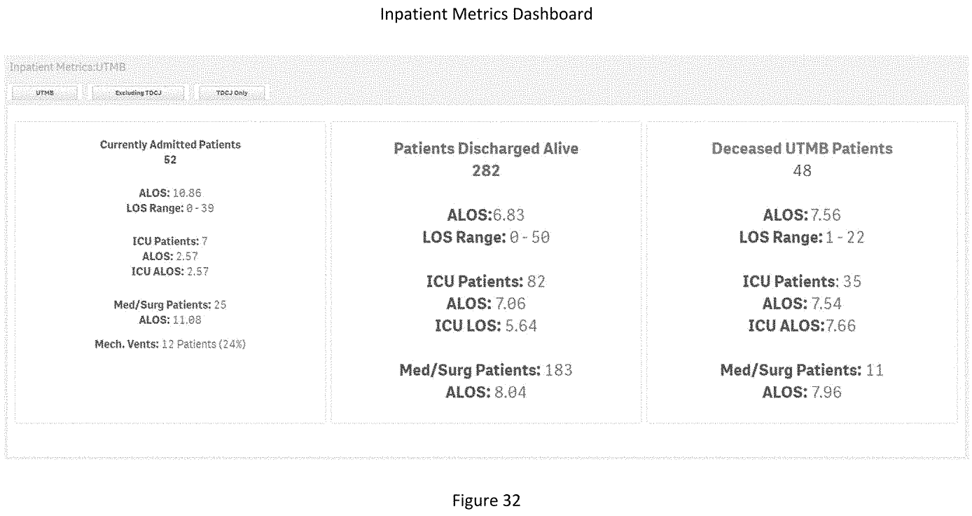

FIG. 32—Inpatient Metrics Dashboard, which displays currently admitted patients (left), patients discharged alive (middle), and deceased UTMB patients (right). Each set of data include ALOS, LOS range, ICU patients, ALOS, ICU ALOS, medical/surgery patients, and ALOS.

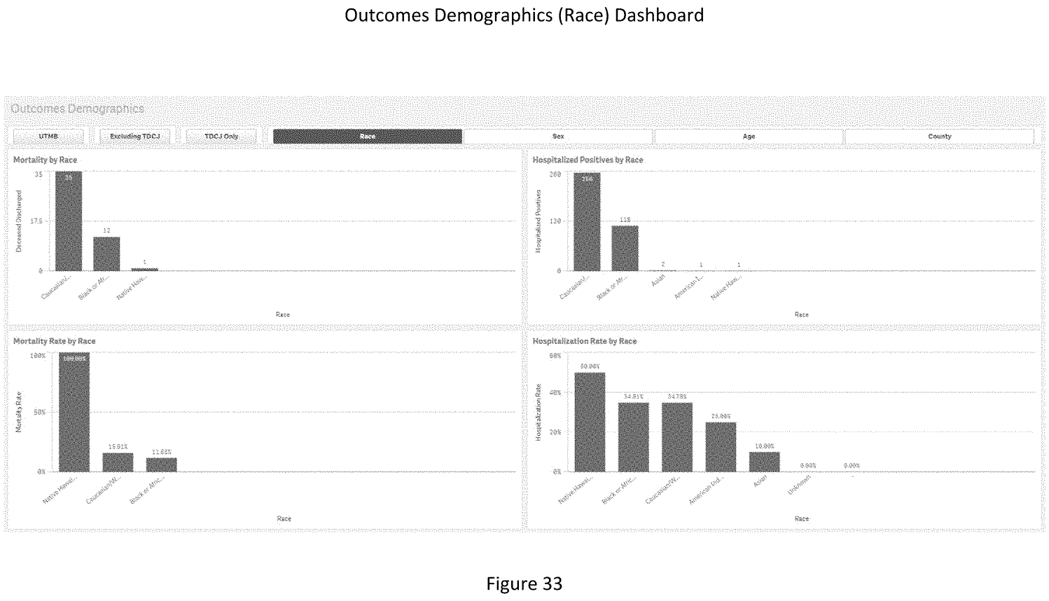

FIG. 33—Outcomes Demographics (Race) Dashboard, which displays graphs for mortality by race (upper left), mortality by race (lower left), hospitalized positives by race (upper right), and hospitalization rate by race (lower right).

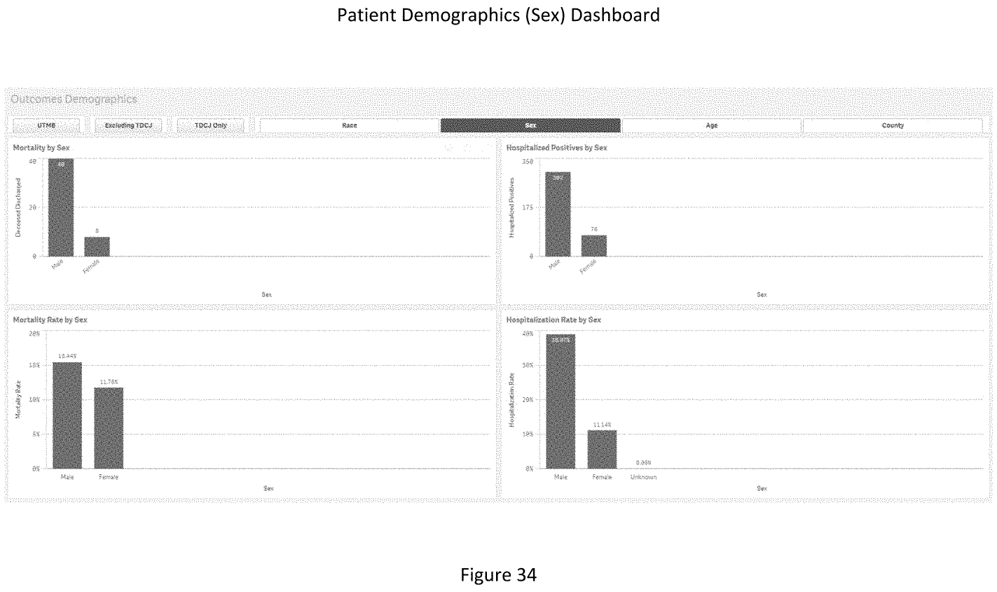

FIG. 34—Outcomes Demographics (Sex) Dashboard, which displays graphs for mortality by sex (upper left), mortality by sex (lower left), hospitalized positives by sex (upper right), and hospitalization rate by sex (lower right).

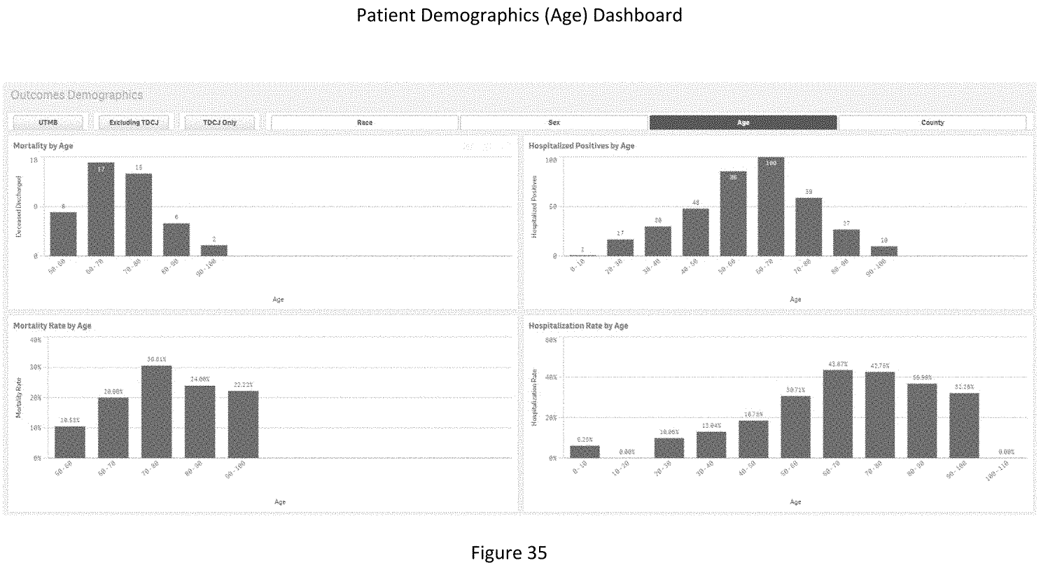

FIG. 35—Outcomes Demographics (Age) Dashboard, which displays graphs for mortality by age (upper left), mortality by age (lower left), hospitalized positives by age (upper right), and hospitalization rate by age (lower right).

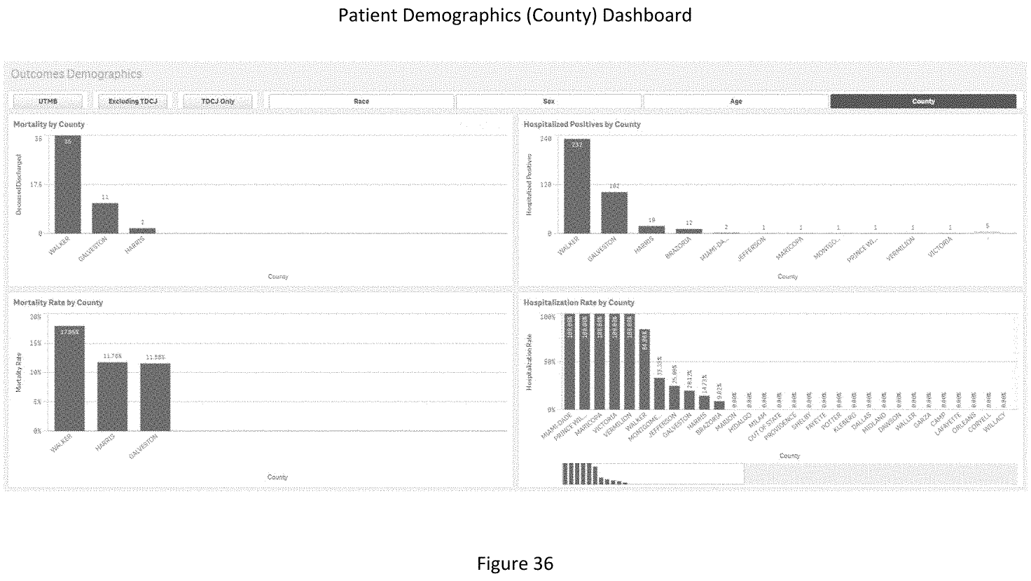

FIG. 36—Outcomes Demographics (County) Dashboard, which displays graphs for mortality by county (upper left), mortality by county (lower left), hospitalized positives by county (upper right), and hospitalization rate by county (lower right).

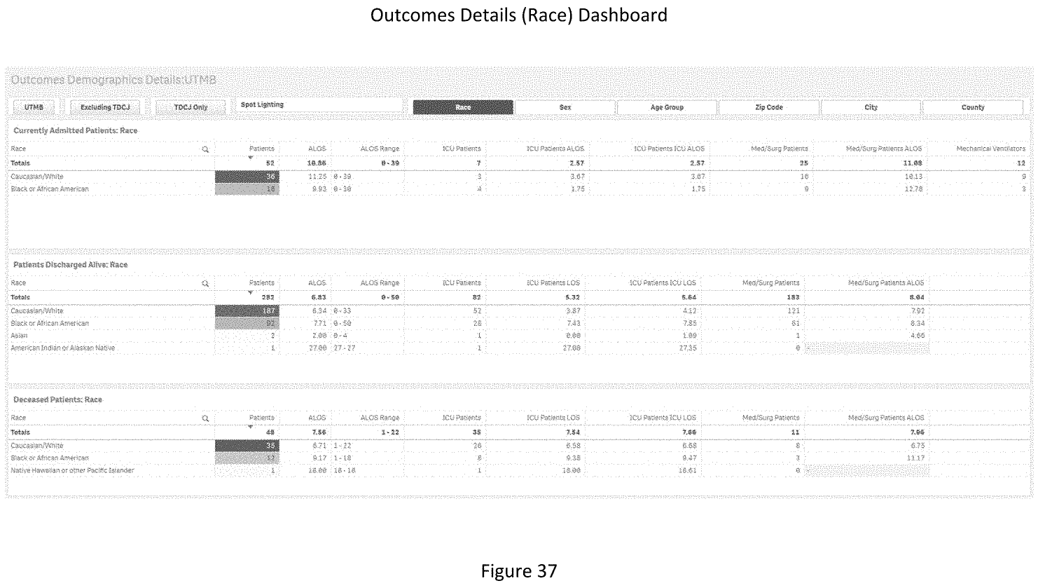

FIG. 37—Outcomes Details (Race) Dashboard, which displays currently admitted patients by race, patients discharged alive by race, and deceased patients by race.

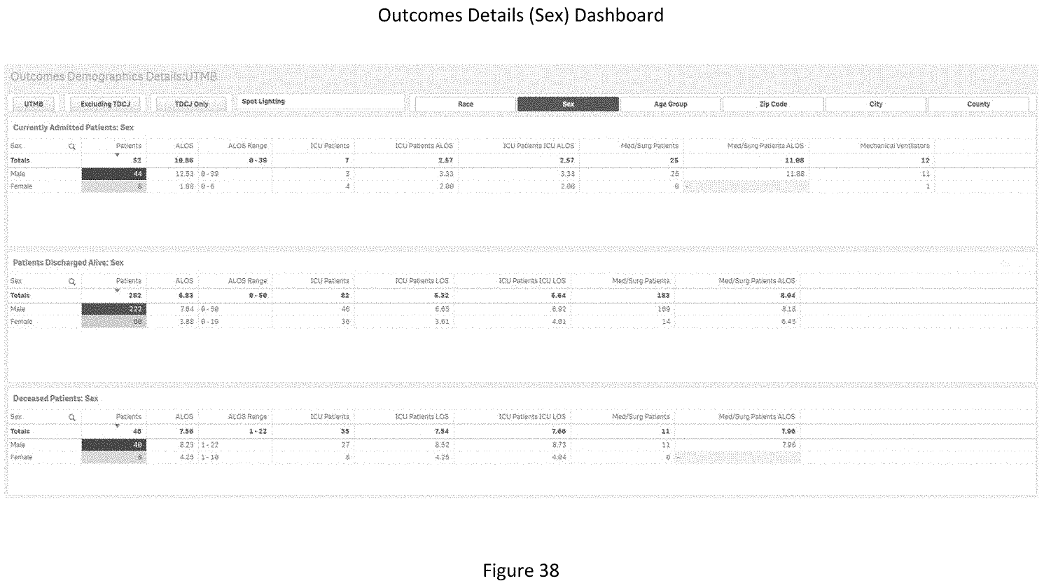

FIG. 38—Outcomes Details (Sex) Dashboard, which displays currently admitted patients by sex, patients discharged alive by sex, and deceased patients by sex.

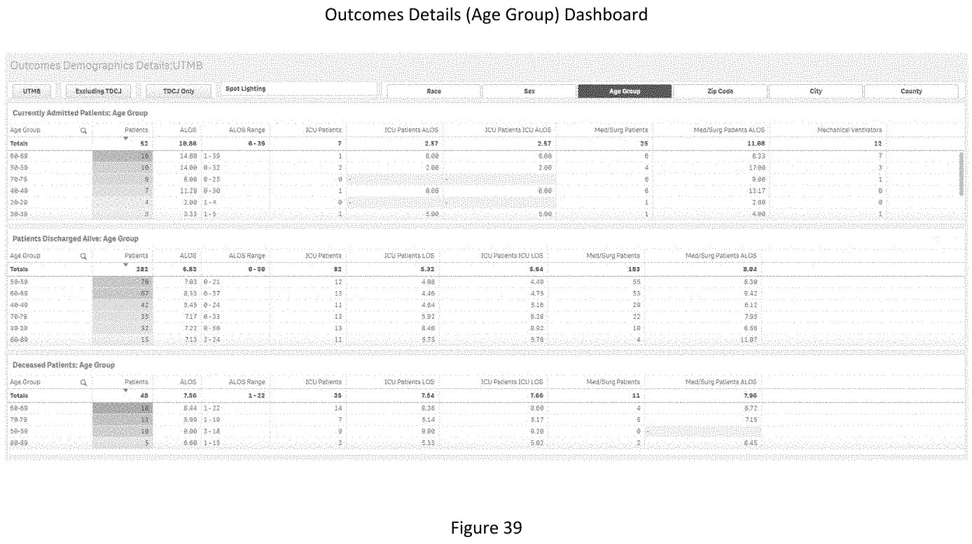

FIG. 39—Outcomes Details (Age Group) Dashboard, which displays currently admitted patients by age, patients discharged alive by age, and deceased patients by age.

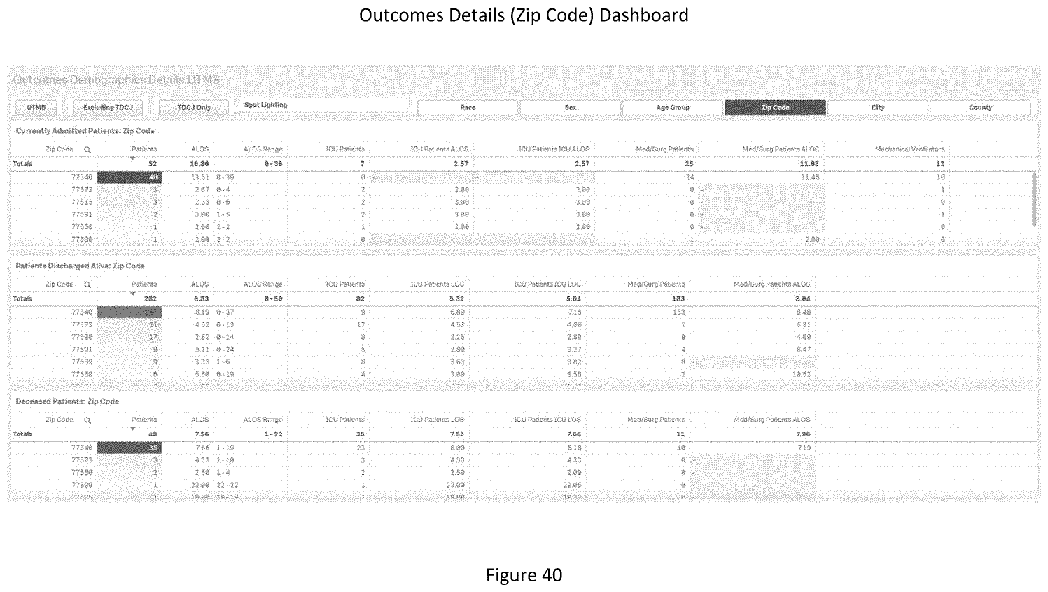

FIG. 40—Outcomes Details (Zip Code) Dashboard, which displays currently admitted patients by zip code, patients discharged alive by zip code, and deceased patients by zip code.

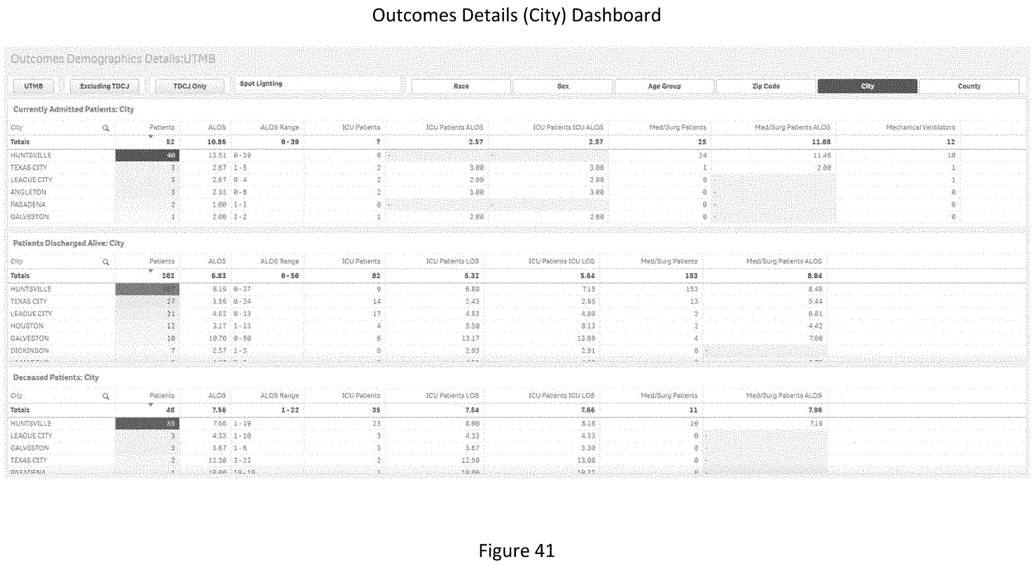

FIG. 41—Outcomes Details (City) Dashboard, which displays currently admitted patients by city, patients discharged alive by city, and deceased patients by city.

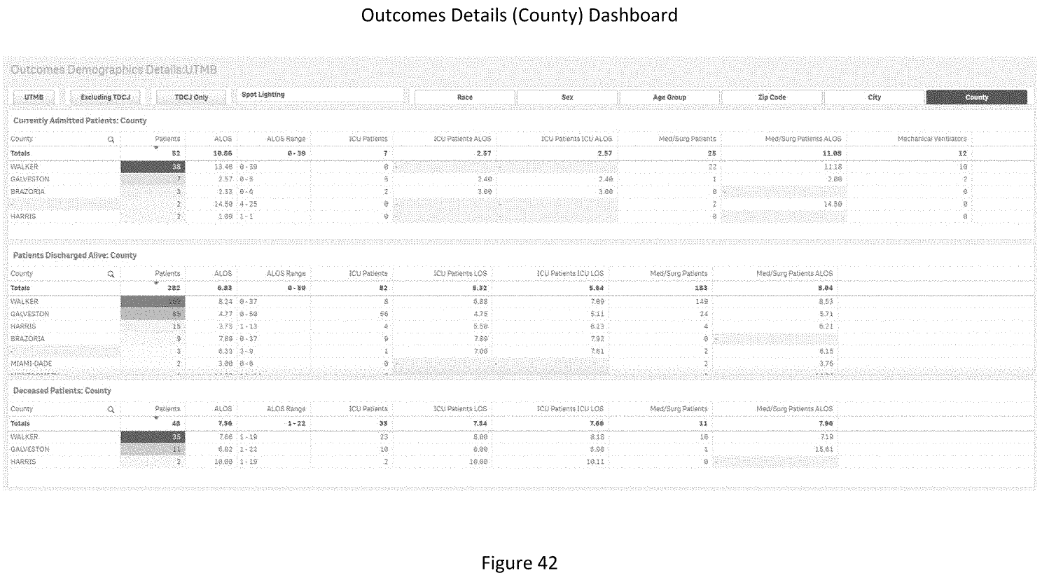

FIG. 42—Outcomes Details (County) Dashboard, which displays currently admitted patients by county, patients discharged alive by county, and deceased patients by county.

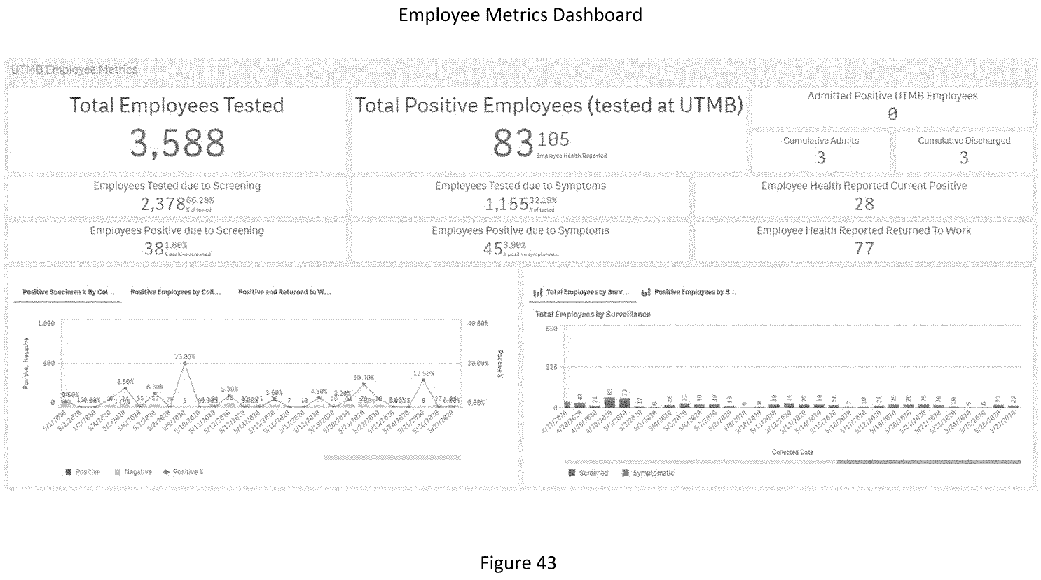

FIG. 43—Employee Metrics Dashboard, which is also described above in reference to FIG. 8.

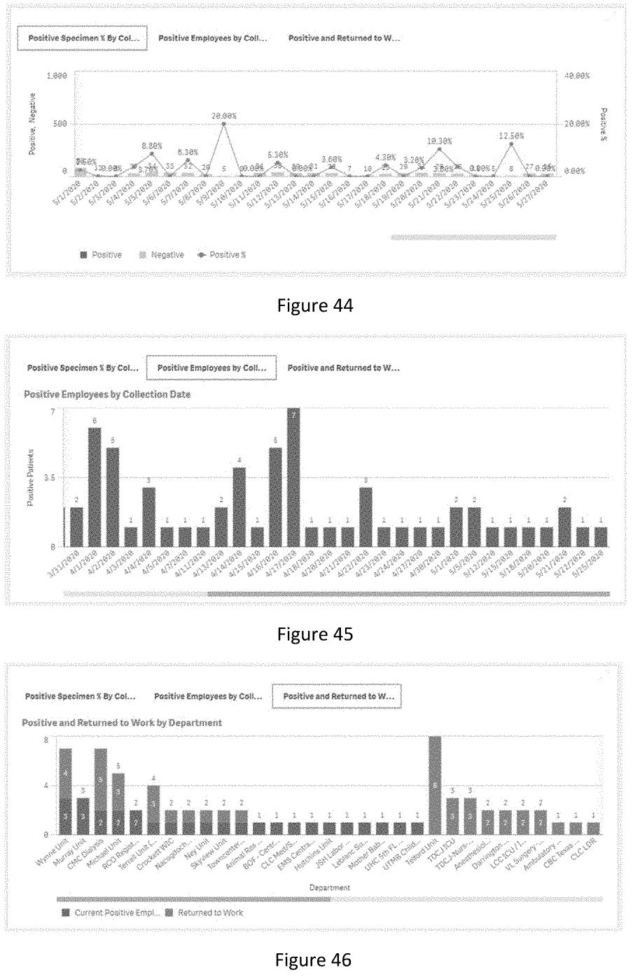

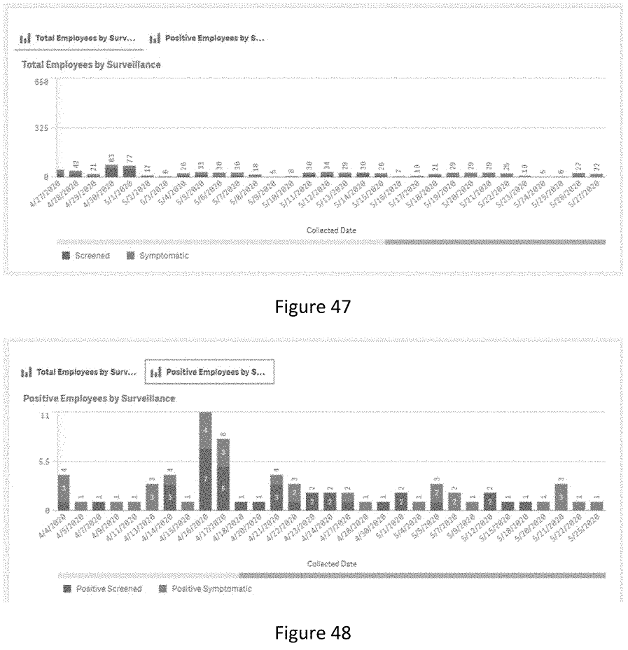

FIGS. 44-48—Employee Metrics Charts: positive specimen % by collection date (FIG. 44), positive employees by collection date (FIG. 45), positive and returned to work by department (FIG. 46), total employees by surveillance (FIG. 47), and positive employees by surveillance (FIG. 48).

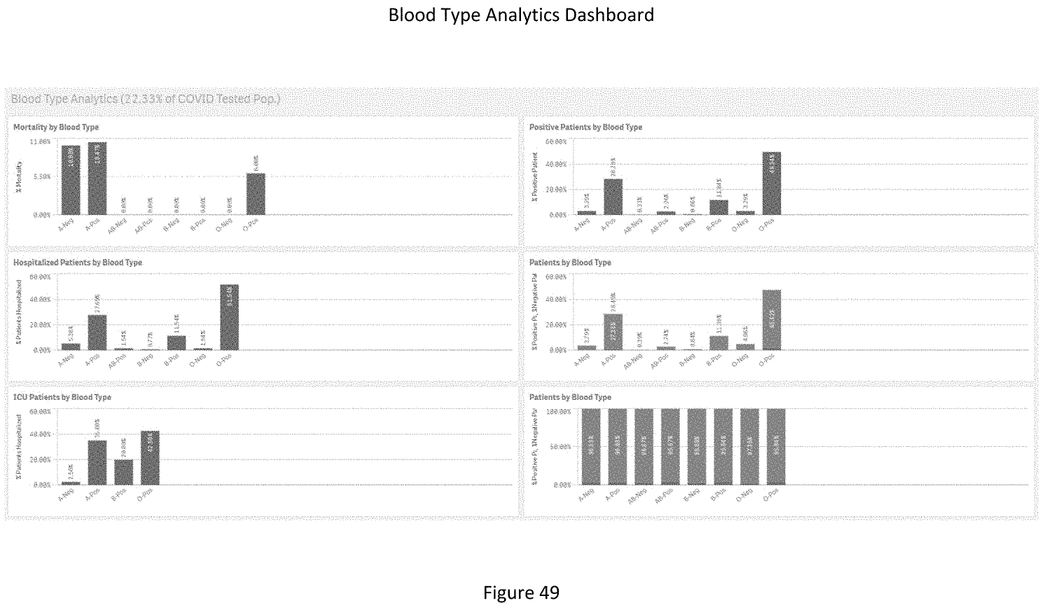

FIG. 49—Blood Type Analytics Dashboard, which displays graphs for mortality by blood type (top left), hospitalized patients by blood type (middle left), ICU patients by blood type (bottom left), positive patients by blood type (upper right), patients by blood type (middle right), and patients by blood type (lower right).

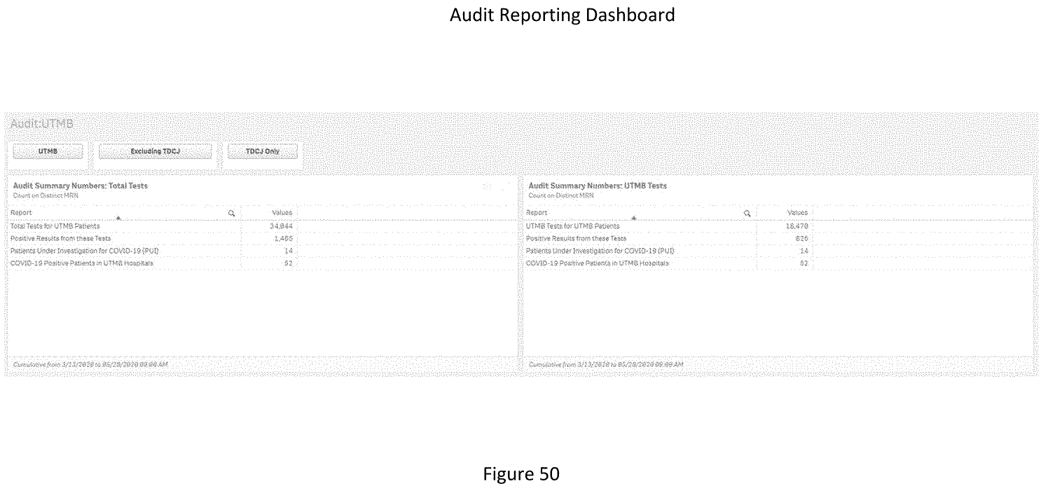

FIG. 50—Audit Reporting Dashboard displays audit summary numbers: total tests (left), and audit summary numbers: UTMB tests (right).

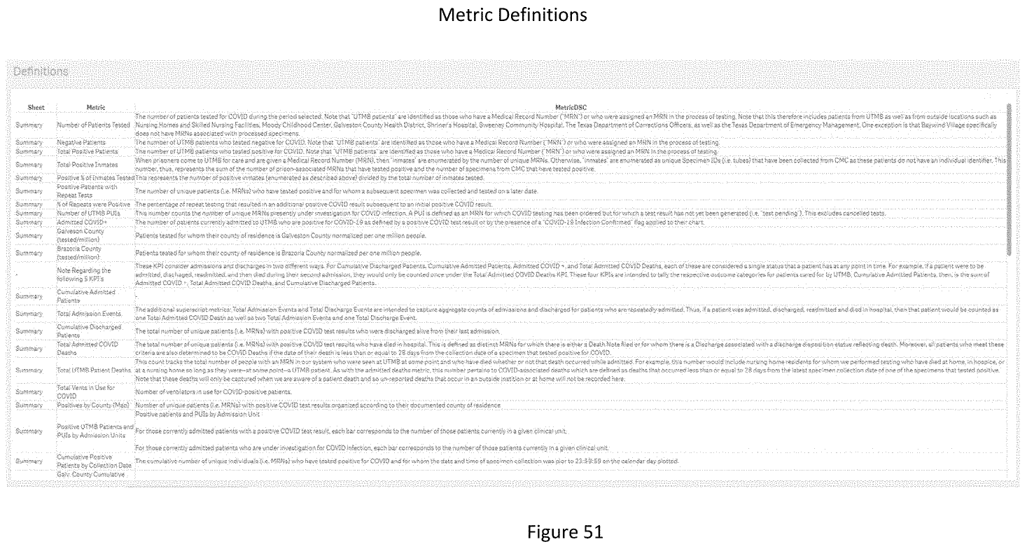

FIG. 51 lists various non-limiting metric definitions.

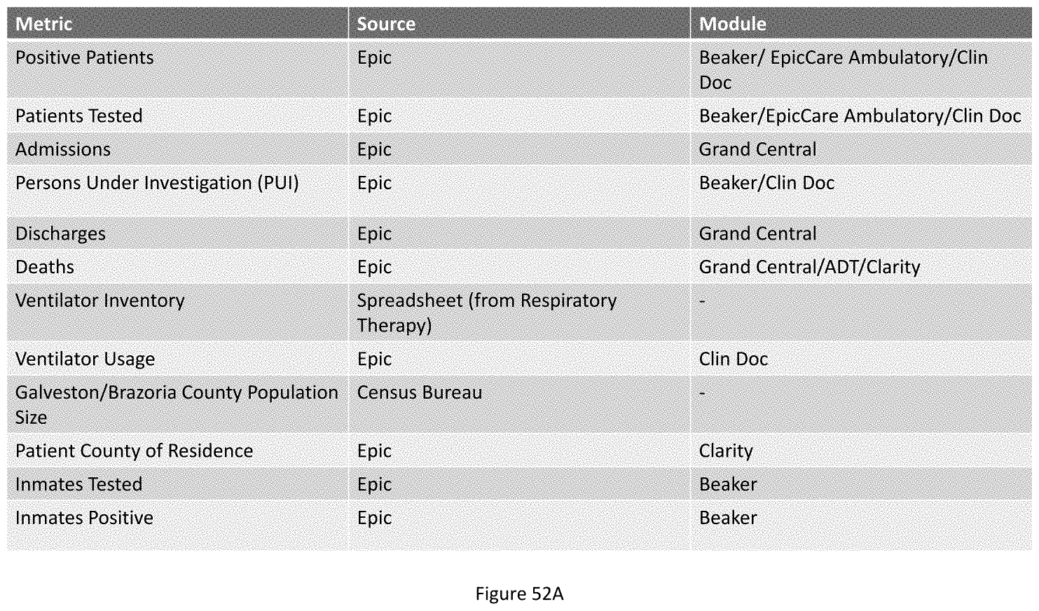

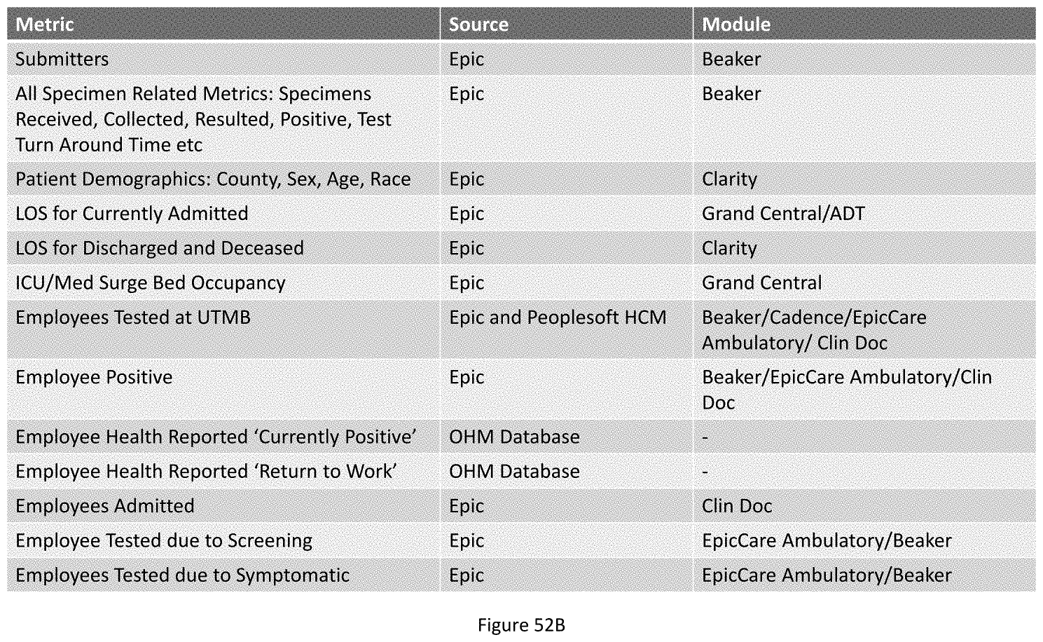

FIGS. 52A-52B list various non-limiting metrics, sources and modules.

To facilitate the understanding of this invention, a number of terms are defined below. Terms defined herein have meanings as commonly understood by a person of ordinary skill in the areas relevant to the present invention. Note that these terms may be used interchangeably without limiting the scope of the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

All of the systems, devices, computer programs, compositions and/or methods disclosed and claimed herein can be made and executed without undue experimentation in light of the present disclosure. While the systems, devices, computer programs, compositions and methods of this invention have been described in terms of preferred embodiments, it will be apparent to those of skill in the art that variations may be applied to the systems, devices, computer programs, compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A computerized method of tracking a disease, comprising:
providing an input/output interface, a memory, a staging database, a relational database and one or more processors communicably coupled to the input/output interface, the memory, the staging database and the relational database;
obtaining a first data corresponding to a first time period for the disease from one or more data sources;
storing the first data in the staging database;
obtaining a second data corresponding to a second time period for the disease from the one or more data sources, wherein the second time period different than the first time period;
storing the second data in the staging database;
processing the first and second data stored in the staging database using a disease curated data model;
storing the processed first and second data in the relational database; and
tracking the disease based on a plurality of metrics using the processed first and second data.

2. The method of claim 1, wherein the first time period is hourly and the second time period is daily.

3. The method of claim 1, wherein tracking the disease further comprises displaying one or more of the plurality of metrics.

4. The method of claim 1, wherein tracking the disease further comprises displaying one or more data visualizations based on one or more of the plurality of metrics or the processed first and second data.

5. The method of claim 1, further comprising automatically sending one or more alerts, guidelines, orders, or recommendations based one or more of the plurality of metrics.

6. The method of claim 1, further comprising automatically allocating or ordering one or more resources based on one or more of the plurality of metrics.

7. The method of claim 1, further comprising using one or more of the plurality of metrics to modify clinic operations, testing protocols, resource allocation or inventory management.

8. The method of claim 1, further comprising predicting one or more metrics at a future time period using the processed first and second data and the disease curated data model.

9. The method of claim 1, further comprising:
receiving a query from a remote communications or processing device;
updating one or more of the plurality of metrics based on the query; and
providing the updated metrics to the remote communications or processing device.

10. The method of claim 1, further comprising:
periodically updating the first data or the second data; and
tracking the disease based on the updated first data or the updated second data.

11. The method of claim 1, wherein the first data or the second data comprises new test orders, test results, admissions, locations, discharges, deaths, patient bed occupancy, ICU length of stay, or ventilator usage.

12. The method of claim 1, wherein the one or more metrics comprise patient metrics, specimen metrics, facility metrics or employee metrics.

13. The method of claim 12, wherein:
the patient metrics comprise positive patients, patients tested, admissions, persons under investigation, discharges, deaths, patient demographics, or patient type;
the specimen metrics comprise submitters, specimens received, specimens collected, specimens resulted, specimens positive, specimens negative or test turn around time;
the facility metrics comprise bed occupancy, occupancy type, length of stay, ventilator usage, discharged, death, transfer; or
the employee metrics comprise employees tested, employees tested positive, employee demographics, employees currently positive, employees returned to work, employees admitted, employees tested due to screening or employees tested due to symptomatic.

14. A system tracking a disease, comprising:
an input/output interface;
a memory;
a staging database;
a relational database;
one or more processors communicably coupled to the input/output interface, the memory, the staging database and the relational database; and
wherein the one or more processors obtain a first data corresponding to a first time period for the disease from one or more data sources, store the first data in the staging database, obtain a second data corresponding to a second time period for the disease from the one or more data sources, wherein the second time period different than the first time period, store the second data in the staging database, process the first and second data stored in the staging database using a disease curated data model, store the processed first and second data in the relational database, and track the disease based on a plurality of metrics using the processed first and second data.

15. The system of claim 14, wherein the first time period is hourly and the second time period is daily.

16. The system of claim 14, wherein one or more processors track the disease by further displaying one or more of the plurality of metrics.

17. The system of claim 14, wherein one or more processors track the disease by further displaying one or more data visualizations based on one or more of the plurality of metrics or the processed first and second data.

18. The system of claim 14, wherein one or more processors automatically sending one or more alerts, guidelines, orders, or recommendations based one or more of the plurality of metrics.

19. The system of claim 14, wherein one or more processors automatically allocating or ordering one or more resources based on one or more of the plurality of metrics.

20. The system of claim 14, wherein one or more processors use one or more of the plurality of metrics to modify clinic operations, testing protocols, resource allocation or inventory management.

21. The system of claim 14, wherein one or more processors predict one or more metrics at a future time period using the processed first and second data and the disease curated data model.

22. The system of claim 14, wherein one or more processors:
receive a query from a remote communications or processing device;
update one or more of the plurality of metrics based on the query; and
provide the updated metrics to the remote communications or processing device.

23. The system of claim 14, wherein one or more processors:
periodically update the first data or the second data; and
track the disease based on the updated first data or the updated second data.

24. The system of claim 14, wherein the first data or the second data comprises new test orders, test results, admissions, locations, discharges, deaths, patient bed occupancy, ICU length of stay, or ventilator usage.

25. The system of claim 14, wherein the one or more metrics comprise patient metrics, specimen metrics, facility metrics or employee metrics.

26. The system of claim 25, wherein:
the patient metrics comprise positive patients, patients tested, admissions, persons under investigation, discharges, deaths, patient demographics, or patient type;
the specimen metrics comprise submitters, specimens received, specimens collected, specimens resulted, specimens positive, specimens negative or test turn around time;
the facility metrics comprise bed occupancy, occupancy type, length of stay, ventilator usage, discharged, death, transfer; or
the employee metrics comprise employees tested, employees tested positive, employee demographics, employees currently positive, employees returned to work, employees admitted, employees tested due to screening or employees tested due to symptomatic.
